(19) United States
(12) Patent Application Publication
(10) Pub. No.: US 2016/0237159 A1
(43) Pub. Date: Aug. 18, 2016

(54) METHODS AND COMPOSITIONS FOR REGULATORY T-CELL ABLATION

(71) Applicant: SLOAN-KETTERING INSTITUTE FOR CANCER RESEARCH, New york, NY (US)

(72) Inventors: Paula D. Bos, New York, NY (US); Alexander Rudensky, New York, NY (US)

(21) Appl. No.: 15/028,531

(22) PCT Filed: Oct. 10, 2014
(86) PCT No.: PCT/US2014/060069

§ 371 Date: Apr. 11, 2016

Related U.S. Application Data
(60) Provisional application No. 61/889,969, filed on Oct. 11, 2013.

(51) Int. Cl. C07K 16/28; C07K 16/30; A61K 45/06; A61K 39/395

(52) U.S. Cl. CPC C07K 16/2866; A61K 39/39558; C07K 16/3015; C07K 16/3023; C07K 16/3053; A61K 45/06; A61K 2039/505

(57) ABSTRACT

The present invention provides, among other things, methods and compositions for the treatment of cancer. The present invention is based, in part, on the surprising discovery that ablation of regulatory T cells (Treg), for example, transient ablation of Treg, is able to drastically reduce tumor burden and reduce metastasis when used as a single agent. In some embodiments, provided methods and compositions are used in combination with one or more other anti-tumor therapies, for example, ionizing radiation.

### CROSS REFERENCE TO RELATED APPLICATIONS

This application claims priority to U.S. Provisional Patent Application No. 61/889,969 filed on Oct. 11, 2013, the contents of which are herein incorporated by reference.

### BACKGROUND

Cancer is one of the most prevalent and treatment-resistant groups of diseases known. While there are hundreds of different cancers, each involves unregulated cell growth with some features that are suggestive of malignancy, which may result in a variety of symptoms and pathologies. In general, cancers are known to have some or all of the following characteristics: sustained proliferative signaling, evasion of growth suppression, resistance to cell death, replicative immortality, induction of angiogenesis, and/or activating invasion and metastasis.

### SUMMARY OF THE INVENTION

The present invention provides, among other things, methods and compositions for the treatment of cancer. The present invention is based, in part, on the surprising discovery that ablation of regulatory T cells (Treg), for example, transient ablation of Treg, is able to drastically reduce tumor burden and reduce metastasis when used as a single agent. In some embodiments, provided methods and compositions are used in combination with one or more other anti-tumor therapies, for example, ionizing radiation. As demonstrated in the Examples below, even transient ablation of Treg is sufficient to significantly reduce tumor burden and metastasis, and significantly prolong survival.

In some embodiments, the present invention provides methods of treating cancer including ablating regulatory t-cells (Treg) in a subject who is suffering from or susceptible to cancer. In some embodiments, the step of ablating comprises administering a Treg ablating agent. In some embodiments, a Treg ablating agent is or comprises a CCR4 antibody or diphtheria toxin (DT).

In some embodiments, the majority of Treg cells are ablated in a subject. In some embodiments, greater than 50%, 60%, 70%, 80%, 90%, 95%, or 99% Treg are ablated in a subject, inclusive. In some embodiments, ablation of Treg is a transient ablation. In some embodiments, Treg are ablated for a period of time equal to or greater than 6 hours, 12 hours, 1 day, 3 days, 1 week, 2 weeks, or one month.

It is contemplated that a variety of dosing regimen may be used in accordance with various embodiments. In some embodiments, the step of ablating comprises administering at least two doses of a Treg ablating agent, separated by a period of time. In some embodiments, the step of ablating comprises administering at least three, four, five, six or more than six doses of a Treg ablating agent, each separated by a period of time. In some embodiments, the period of time between each administration is the same. In some embodiments, the period of time between each administration is different. In some embodiments, the period of time between doses may be 1 hour, 2 hours, 3 hours, 6 hours, 12 hours, 24 hours, 48 hours, 72 hours, 96 hours, 1 week, 2 weeks, or 1 month. In some embodiments the period of time between doses is greater than 1 month.

According to various embodiments comprising administration of two or more doses of a Treg ablating agent, the dose of Treg ablating agent may vary according to sound medical judgment. In some embodiments, each dose of a Treg ablating agent is the same. In some embodiments, each dose of a Treg ablating agent may vary from one or more other doses.

According to several embodiments, ablation of Treg results in a decrease in tumor burden in a subject as compared to the tumor burden of the subject pre-treatment. In some embodiments, ablation of Treg results in a reduction of tumor burden of at least 30%, 40%, 50%, 60%, 70%, 80%, 90%, or 95%, inclusive as compared to the tumor burden of the subject pre-treatment.

It is contemplated that provided methods and compositions may be used to treat any of a variety of cancers. In some embodiments, the cancer is metastatic. In some embodiments, the cancer is a solid tumor. In some embodiments, the cancer comprises a primary tumor. In some embodiments, the cancer comprises a secondary tumor. In some embodiments, the cancer is selected from the group consisting of: breast cancer, prostate cancer, melanoma, renal cell carcinoma, non-small cell lung cancer, and ovarian cancer.

In some embodiments, Treg are Foxp3⁺ t-cells. In some embodiments, ablation of Treg may be verified and/or quantified through detection of a decreased number of Foxp3⁺ cells. In some embodiments, Treg are Foxp3⁺ CD25⁺ CD4⁺ cells. In some embodiments, ablation of Treg may be verified and/or quantified through detection of a decreased number of Foxp3⁺ CD25⁺ CD4⁺ cells.

In some embodiments, provided methods further include administering to the subject one or more of an anticancer agent and ionizing radiation. In some embodiments, the anti-cancer agent is an anti-CTLA4 agent, an anti-PD-1 agent, and/or an anti-PD-L1 agent. In some embodiments, the anti-cancer agent is selected from the group consisting of surgery, radiotherapy, endocrine therapy, an interferon, an interleukin, tumor necrosis factor (TNF), hyperthermia, cryotherapy, antiemetics, an alkylating drug (e.g., mechlorethamine, chlorambucil, cyclophosphamide, melphalan, ifosfamide), antimetabolites (e.g., methotrexate), purine antagonists and pyrimidine antagonists (e.g., 6-Mercaptopurine, 5-Fluorouracil, Cytarabile, Gemcitabine), spindle poisons (e.g., vinblastine, vincristine, vinorelbine, paclitaxel), podophyllotoxins (e.g., etoposide, irinotecan, topotecan), antibiotics (e.g., doxorubicin, bleomycin, mitomycin), nitrosoureas (e.g., carmustine, lomustine), inorganic ions (e.g., cisplatin, carboplatin), enzymes (e.g., asparaginase), and hormones (e.g., tamoxifen, leuprolide, flutamide, and megestrol), anastrozole, letrozole, erlotinib, iressa, tarceva, gemcitabine, doxorubicin, cyclophosphamide, gemcitabine, adriamycin, and trastuzumab and/or any other approved chemotherapeutic drug(s).

In some embodiments, the amount of ionizing radiation administered is between about 1 Gy and about 1,000 Gy, about 5 Gy and about 900 Gy, about 10 Gy to about 800 Gy, about 10 Gy to about 700 Gy, about 10 Gy to about 600 Gy, about 10 Gy to about 500 Gy, about 10 Gy to about 400 Gy, about 10 Gy to about 300 Gy, about 10 Gy to about 200 Gy, about 10 Gy to about 100 Gy, about 5 Gy and about 15 Gy, between about 7.5 Gy and about 12 Gy, or between about 10 Gy and about 12 Gy. In some embodiments, the amount of ionizing radiation administered is about 12 Gy. In some embodiments, the amount of ionizing radiation is greater than about 10, 20, 30, 40, 50, 60, 70, 80, 90, 100, 200, 300, 400, 500, or 1,000 Gy. In some embodiments, the amount of ionizing radiation is less than about 1,000, 900, 800, 700, 600, 500, 400, 300, 200, 100, 90, 80, 70, 60, or 50 Gy.

In some embodiments, the ablation of Treg results in at least one symptom or feature of cancer being reduced in intensity, severity, duration, or frequency, and/or has delayed in onset.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### DEFINITIONS

In order for the present invention to be more readily understood, certain terms are first defined below. Additional definitions for the following terms and other terms are set forth throughout the specification.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Cancer: As used herein, the term “cancer” refers to a group of diseases, all involving unregulated cell growth. Exemplary cancers include, without limitation: Acute lymphoblastic leukemia, Acute myeloid leukemia, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liposarcoma; Liver Cancer (Primary); Lung Cancer, Non-Small Cell; Lung Cancer Small Cell Lymphomas; Lymphoma, Burkitt; Lymphoma, cutaneous T-Cell; Lymphoma, Hodgkin; Lymphoma, Primary Central Nervous System; Macroglobulinemia, Waldenström; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sézary syndrome; Skin cancer (nonmelanoma); Skin carcinoma, Merkel cell; Small intestine cancer; Soft tissue sarcoma; Squamous cell carcinoma; Squamous neck cancer with occult primary, metastatic; Stomach cancer; Supratentorial primitive neuroectodermal tumor, childhood; T-Cell lymphoma, cutaneous; Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; and Wilms tumor (kidney cancer), childhood.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g., phosphate-buffered saline), sterile saline solution, Ringer's solution or dextrose solution.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Immunotherapy: as used herein the term “immunotherapy” refers to the treatment of disease by inducing, enhancing, or suppressing an immune response. The immune response may be active or passive, the response may be a Th1 or Th2 response, or take any other form as appropriate for a particular application of the invention.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Prevent: As used herein, the term “prevent” or “prevention”, when used in connection with the occurrence of a disease, disorder, and/or condition, refers to reducing the risk of developing the disease, disorder and/or condition. See the definition of “risk.”

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Risk: As will be understood from context, a “risk” of a disease, disorder, and/or condition comprises a likelihood that a particular individual will develop a disease, disorder, and/or condition (e.g., cancer). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., cancer). In some embodiments a reference sample or group of reference samples are from individuals comparable to a particular individual. In some embodiments, relative risk is 0, 1, 2, 3, 4, 5, 6, 7, 8, 9, 10, or more.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, condition, or event (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Tumor Burden: As used herein, the term “tumor burden” refers to the total mass of tumor tissue carried by an individual with cancer. The total mass of tumor tissue may be quantified according to any medically appropriate scheme, for example, by measuring the size of tumor(s) or through counting or approximating the number of cancer cells in a patient.

### BRIEF DESCRIPTION OF THE DRAWING

FIG. 1 shows exemplary effects of Treg cell ablation at the time of orthotopic tumor implantation: A) schematic of experimental set up; B) growth kinetics of orthotopic tumors in mice treated with 50 μg/kg DT from the day of tumor progression; C) fraction and exemplary image of mice with detectable lung metastasis upon bioluminescence imaging of the dissected lungs from the group depicted in B; D, E) flow cytometric quantification of intratumoral CD4+ FOXP3+ T cells, and proliferation (ki67+) and activation state of intratumoral CD4+ and CD8+ cells (D) and frequency of CD118B+, immature myeloid cells and IFN-γ production in T cells (E); top: control, bottom: DT-treated.

FIG. 2 shows exemplary effects of Treg cell ablation on the growth of established primary and lung metastatic tumors: A) schematic of the experimental set up, black arrows indicate day of tumor implantation (↓) and analysis (↑); B) growth kinetics of orthotopic tumors in mice treated with 50 μg/kg DT when tumors reached approximately 250 mm³; C) fraction and representative image of mice with detectable lung metastasis upon bioluminescence imaging of the dissected lungs form the group depicted in B; D) histologic quantification and representative H&E staining image of the area of the lungs occupied with tumors in experimental lung colonization experiments.

FIG. 3 shows exemplary results wherein the ablation of Treg cells results in tumor cell death in autochthonous breast tumors: A) frequency of CD4+ Foxp3+ Treg in indicated organs of control and tumor-bearing MMTV-rtTA, tet-O-PyMT (TOMT) mice; B) schedule of DT treatment in TOMT mice; C) flow cytometric quantification of intratumoral CD4+ Foxp3+ Treg cells at end point (10 days after first DT injection); D) histologic quantification and representative images of tumor cell death by cleaved caspase-3 immunohistochemistry; E) flow cytometric determination of the frequency of intratumoral proliferating (ki67+) and naïve CD62L^{high} CD44^{lo} CD4+ and CD8+ T cells; LN=lymph node, NDL=non-draining lymph node, M. Gland=mammary gland.

FIG. 4 shows exemplary: A) growth kinetics of orthotopically implanted tumors treated with 25 μg/kg DT at the indicated times; B) number of lung metastatic nodules present on lung surface upon examination under a dissection microscope; C) weight fluctuations represented as percentage of weight at the time of DT administration; D) representative histological images of liver, kidney, heart and pancreas from control and DT-treated mice 2 weeks after treatment; all images are at 20× magnification.

FIG. 5 depicts exemplary: A) experimental set up of DT treatment and analysis; B) significant changes in DT-treated tumors analyzed by cytokine/chemokine array; C) concentration (in pgml) of IFN-γ, CXCL9, and CXCL10 in control and DT-treated tumors in one representative multiplex assay; D) confirmation of cell-type-specific production of these secreted factors by semi-quantitative PCR, RNA was extracted from CD45⁺ TCRβ⁻ CD11B⁺ Gr1⁻ or CD45⁺ TCRβ⁺ CD11B⁻ FACS-sorted cells; E) semi-quantitative PCR analysis of iNOS.

FIG. 6 depicts exemplary: A-C) growth kinetics of orthotopically implanted tumors in mice treated with 25 μg/kg DT at indicated times and receiving one dose of 1 mg anti-IFN-γ at day 10 (A), one dose of 300 μg anti-Nk1.1 on day 10 (B), or one dose of 250 μg anti-CD8 antibody at day 13 (C); D) tumor growth kinetics of control or DT-treated Foxp3^{DTR} β2M^{-/-} mice.

FIG. 7 shows exemplary data after a single treatment with PD-1 or PD-L1 on CD4⁺ cell in control (top) or DT-treated tumors (bottom) assessed by flow cytometry; B) tumor growth kinetics of orthotopically implanted PyMT carcinoma cells in control or PD-1 (top) or PD-L1 (bottom) antibody treated mice; C) number of lung metastatic nodules in mice treated with PD-L1 antibody.

FIG. 8 shows exemplary effects of checkpoint blockade on oncogene-driven tumor growth and lung metastasis: A) diagram of experimental set up, purple arrows indicated injection of 25 μg/kg of DT, blue arrows indicate injection of specific antibody, and black arrows indicated day of tumor implantation; B, D) tumor growth kinetics of orthotopically implanted PyMT-driven mammary carcinomas, mice treated with 0.1 mg CTLA-4 (B) or 0.25 mg PD-1+0.1 mg PD-L1 antibodies (D) at day 0, 3 and 6 after tumors reached approximately 100 mm³; C, E) number of lung metastatic nodules present on the lung surface upon examination under a dissecting microscope.

FIG. 9 shows exemplary ionizing radiation (IR) dose determination and Treg radioresistance: A) ratio of Treg/CD4 T cells; B) ratio of Treg/CD8 T cells, in control and irradiated tumors 1, 2 and 4 days after radiotherapy; C) radiation dose-dependent effects on MMTV-PyMT orthotopic tumor cell growth.

FIG. 10 shows exemplary: A) diagram of experimental set up; B) tumor growth kinetics of mice receiving radiation alone, DT alone, a combination of both, or no treatment; C) analysis of fold change increase in tumor size for each group at day 27 after initial treatment; D) day at which a given tumor reaches at least 1,000 mm³; E) survival analysis of mice in each of the previously described groups; F) time-matched quantification of lung metastatic nodules in lungs from mice in each treatment group.

FIG. 11 shows: A) representative images of histological staining with cleaved Caspase-3 (cC3), depicting the area of apoptotic cells observed in each individual tumor; B) quantification of cC3 staining in healthy areas of the tissue; C) histological determination of the number of CD45⁺ IBA1⁺ cells in representative regions of the tumor; D) increase influx in CD115⁺ intratumoral leukocytes.

FIG. 12 shows A) Growth kinetics of orthotopic B16-ova melanoma tumors in Foxp3-DTR mice treated with DT when tumors reached approximately 100 mm³. B) Survival curves of Foxp3-DTR mice with orthotopic B16 melanoma tumors treated with DT or vehicle control C) Flow cytometric quantification of peripheral antigen specific CD8+ OVAtet+ T cells.

FIG. 13 shows A) A plot derived from flow cytometric analysis of Foxp3-expressing cells within the CD4+ T cell compartment in lungs from uninjected control mice and from Lewis Lung Carcinoma (LLC) tumors showing increased percentages of Foxp3+ cells in tumors. B) Tumor burden measured 24 days injection of LLC cells into Foxp3-DTR mice which were treated with diptheria toxin (DT) to ablate Treg cells and/or paclitaxel (10 mg/kg) at days 10, 13, and 16 after injection.

### DETAILED DESCRIPTION

The present invention provides, among other things, methods of treating cancer including ablating regulatory t-cells (Treg) in a subject who is suffering from or susceptible to cancer. In part, the present invention is based on the surprising discovery that ablation of Treg, even a transient ablation, results in a significant reduction in tumor burden and/or metastasis in a subject, as well as a significant increase in survival. Provided methods and compositions are able to produce dramatic effects alone or in combination with one or more other anti-cancer agents or therapies.

Various aspects of the invention are described in detail in the following sections. The use of sections is not meant to limit the invention. Each section can apply to any aspect of the invention. In this application, the use of “or” means “and/or” unless stated otherwise.

Regulatory T Cells

Regulatory T cells (Treg) are important in maintaining homeostasis, controlling the magnitude and duration of the inflammatory response, and in preventing autoimmune and allergic responses. There are two major classifications of Treg: natural Treg and induced Treg. Natural Treg, (nTreg) are a class of thymically generated T-cells while induced Treg (iTreg) develop in the periphery from naïve T cells in response to signals such as low doses of antigen, presence of certain microbes, lymphopenia or, in some cases, through activation by immature dendritic cells. In some cases, iTreg are thought to be generated in response to inflammatory conditions, particularly those which may be due at least in part to the absence of nTreg cells.

The Forkhead box P3 transcription factor (Foxp3) has been shown to be a key regulator in the differentiation and activity of Treg. In fact, loss-of-function mutations in the Foxp3 gene have been shown to lead to the lethal IPEX syndrome (immune dysregulation, polyendocrinopathy, enteropathy, X-linked). Patients with IPEX suffer from severe autoimmune responses, persistent eczema, and colitis.

In general Treg are thought to be mainly involved in suppressing immune responses, functioning in part as a “self-check” for the immune system to prevent excessive reactions. In particular, Treg are involved in maintaining tolerance to self-antigens, harmless agents such as pollen or food, and abrogating autoimmune disease.

Treg are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, Regulatory T cells control immune responses through their non-redundant tissue specific features, 2013, FRONTIERS IN IMMUNOL., 4(294): 1-10, the disclosure of which is hereby incorporated in its entirety.

Typically, regulatory T cells are known to require TGF-β and IL-2 for proper activation and development. Blockade of TGF-β signaling has been shown to result in systemic inflammatory disease as a result of a deficiency of Treg and IL-2 knockout mice have been shown to fail to develop Treg. TGF-β may be particularly important, as it is known to stimulate Foxp3, the transcription factor that drives differentiation of T cells toward the Treg lineage.

Regulatory T cells are known to produce both IL-10 and TGF-β, both potent immune suppressive cytokines Additionally, Treg are known to inhibit the ability of antigen presenting cells (APCs) to stimulate T cells. One proposed mechanism for APC inhibition is via CTLA-4, which is expressed by Foxp3⁺ Treg. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Treg may be found in Dhamne et al., Peripheral and thymic Foxp3+ regulatory T cells in search of origin, distinction, and function, 2013, Frontiers in Immunol., 4 (253): 1-11, the disclosure of which is hereby incorporated in its entirety.

Treg Ablation

According to various embodiments, provided methods and compositions include one or more Treg ablating agents and/or strategies for Treg ablation. As used herein a “Treg ablating agent” means a substance or method capable of ablating (e.g., depleting) a significant portion of a subject's Treg. In some embodiments, the majority of Treg cells are ablated in a subject. In some embodiments, greater than 50%, 60%, 70%, 80%, 90%, 95%, or 99% Treg are ablated in a subject, inclusive.

In some embodiments, a Treg ablating agent is a biological agent, such as a protein or peptide-based agent. In some embodiments, a protein or peptide based ablating agent targets chemokine receptor type 4 (CCR4). In some embodiments, a Treg ablating agent is a monoclonal or polyclonal antibody to CCR4. In some embodiments, a CCR4 antibody is a humanized antibody.

According to several embodiments, ablation of Treg results in a decrease in tumor burden in a subject as compared to the tumor burden of the subject pre-treatment. In some embodiments, ablation of Treg results in a reduction of tumor burden of at least 30%, 40%, 50%, 60%, 70%, 80%, 90%, or 95%, inclusive as compared to the tumor burden of the subject pre-treatment.

In some embodiments, ablation of Treg results in an increase in survival time of a subject as compared to a statistical average survival time of a subject suffering from the same or a similar cancer. In some embodiments, ablation of Treg results in an increase in survival time of 10%, 20%, 30%, 40%, 50%, 60%, 70%, 80%, 90%, or 95% or more as compared to a statistical average survival time of a subject suffering from the same or a similar cancer. In some embodiments, the increase in survival time may be 1 month, 3 months, 6 months, 1 year, 2 years, 5 years, or more as compared to a statistical average survival time of a subject suffering from the same or a similar cancer.

According to various embodiments, Treg are transiently ablated. In some embodiments, Treg are ablated for a period of time equal to or greater than 1 hour, 3 hours, 6 hours, 12 hours, 1 day, 3 days, 1 week, 2 weeks, or one month.

In some embodiments, one or more tests are performed to verify and/or quantitate the degree of Treg ablation. In some embodiments, ablation of Treg may be verified and/or quantified through detection of a decreased number of Foxp3⁺ cells. In some embodiments, ablation of Treg may be verified and/or quantified through detection of a decreased number of Foxp3⁺ CD25⁺ CD4⁺ cells.

In some embodiments, the ablation of Treg results in at least one symptom or feature of cancer being reduced in intensity, severity, duration, or frequency, and/or has delayed in onset.

In some embodiments, the present invention provides methods and systems for identifying and/or characterizing Treg ablating agents and/or protocols. In some embodiments, provided methods and systems include administering one or more candidate Treg ablating agents and/or protocols to a population of Treg and assaying for cell survival and/or proliferation. In some embodiments, the population of Treg is an in vitro population. In some embodiments, the Treg population is an in vivo population. In some embodiments, a candidate Treg ablating agent and/or protocol is considered a Treg ablating agent and/or protocol if administration results in a decrease in Treg population by 10%, 20%, 30%, 40%, 50%, 60%, 70%, 80%, 90%, or more as compared to a similar Treg population that was not exposed to the agent(s) and/or protocol(s).

Cancer

Cancer, as used herein, refers to a group of diseases, all of which involve unregulated cell growth. Cancer is generally understood to be a deadly disease. Taken as a whole, about half of people receiving treatment for invasive cancers die from cancer or from effects of treatment. In 2008, approximately 12.7 million cancers were diagnosed (excluding non-melanoma skin cancers and other non-invasive cancers) and 7.6 million people died of cancer worldwide. There is clearly a large unmet need for more successful treatments for cancer.

The causes of cancer are diverse, complex and, for the most part, poorly understood. Many things are known or thought to increase the risk of developing cancer such as tobacco use, dietary factors, exposure to radiation, obesity, and exposure to environmental pollutants, to name a few.

Cancer may be detected in a number of ways, depending upon the type, including, but not limited to screening tests such as blood or urine tests, medical imaging including X-ray, CT, and MRI, and/or the presence of certain signs or symptoms. In some embodiments, signs and symptoms may include one or more of the following: development of an abnormal mass of tissue, which may obstruct or completely block a passage or opening such as the bronchus, esophagus, colon, bladder or uterus; unintentional weight loss; fever; excessive fatigue; persistent unexplained muscle or joint pain, and changes in the coloration and/or appearance of the skin.

Types of cancer include but are not limited to lung cancer, breast cancer, colorectal cancer, prostate cancer, leukemia, lymphoma, non-Hodgkin's lymphoma, skin cancer, brain cancer, cancer of the central nervous system, ovarian cancer, uterine cancer, stomach cancer, pancreatic cancer, esophageal cancer, kidney cancer, liver cancer, or a head and neck cancer.

It is contemplated that provided methods and compositions may be used to treat any of a variety of cancers. In some embodiments, the cancer is metastatic. In some embodiments, the cancer is a solid tumor. In some embodiments, the cancer comprises a primary tumor. In some embodiments, the cancer comprises a secondary tumor. In some embodiments, the cancer is selected from the group consisting of: breast cancer, prostate cancer, melanoma, renal cell carcinoma, non-small cell lung cancer, and ovarian cancer.

Additional non-limiting examples of cancers contemplated as within the scope of the present invention include, but are not limited to: leukemia, such as, but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemia, such as, but not limited to, chronic myelocytic (granulocytic) leukemia, chronic lymphocytic leukemia, hairy cell leukemia; polycythemia vera; lymphoma such as, but not limited to, Hodgkin's disease, non-Hodgkin's disease; multiple myeloma such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; dendritic cell cancer, including plasmacytoid dendritic cell cancer, NK blastic lymphoma (also known as cutaneous NK/T-cell lymphoma and agranular (CD4+/CD56+) dermatologic neoplasms); basophilic leukemia; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; a brain tumor such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as, but not limited to, pheochromocytom and adrenocortical carcinoma; thyroid cancer such as, but not limited to, papillary or follicular thyroid cancer, medullary thyroid cancer and anaplastic thyroid cancer; pancreatic cancer such as, but not limited to, insulinoma, gastrinoma, glucagonoma, vipoma, somatostatin-secreting tumor, and carcinoid or islet cell tumor; pituitary cancer such as, but limited to, Cushing's disease, prolactin-secreting tumor, acromegaly, and diabetes insipius; eye cancer such as but not limited to ocular melanoma such as iris melanoma, choroidal melanoma, and cilliary body melanoma, and retinoblastoma; vaginal cancer such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancer such as but not limited to, squamous cell carcinoma, and adenocarcinoma; uterine cancer such as but not limited to endometrial carcinoma and uterine sarcoma; ovarian cancer such as, but not limited to, ovarian epithelial carcinoma, borderline tumor, germ cell tumor, and stromal tumor; esophageal cancer such as, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancer such as, but not limited to, adenocarcinoma, fungating (polypoid), ulcerating, superficial spreading, diffusely spreading, malignant lymphoma, liposarcoma, fibrosarcoma, and carcinosarcoma; colon cancer; rectal cancer; liver cancer such as, but not limited to, hepatocellular carcinoma and hepatoblastoma; gallbladder cancer such as adenocarcinoma; cholangiocarcinomas such as, but not limited to, papillary, nodular, and diffuse; lung cancer such as non-small cell lung cancer, squamous cell carcinoma (epidermoid carcinoma), adenocarcinoma, large-cell carcinoma and small-cell lung cancer; testicular cancer such as, but not limited to, germinal tumor, seminoma, anaplastic, classic (typical), spermatocytic, nonseminoma, embryonal carcinoma, teratoma carcinoma, choriocarcinoma (yolk-sac tumor), prostate cancer such as, but not limited to, prostatic intraepithelial neoplasia, adenocarcinoma, leiomyosarcoma, and rhabdomyosarcoma; penile cancer; oral cancer such as, but not limited to, squamous cell carcinoma; basal cancer; salivary gland cancer such as, but not limited to, adenocarcinoma, mucoepidermoid carcinoma, and adenoidcystic carcinoma; pharynx cancer such as, but not limited to, squamous cell cancer, and verrucous; skin cancer such as, but not limited to, basal cell carcinoma, squamous cell carcinoma and melanoma, superficial spreading melanoma, nodular melanoma, lentigo malignant melanoma, acral lentiginous melanoma; kidney cancer such as, but not limited to, renal cell carcinoma, adenocarcinoma, hypernephroma, fibrosarcoma, transitional cell cancer (renal pelvis and/or uterer); Wilms' tumor; bladder cancer such as, but not limited to, transitional cell carcinoma, squamous cell cancer, adenocarcinoma, carcinosarcoma. In addition, cancer include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Generally, the cause of cancers are not known. As a result, in some embodiments, it is useful to identify one or more risk factors associated with the development of one or more types of cancer. In some embodiments, this identification may be used to identify subjects at risk for developing one or more cancers. Exemplary risk factors for developing cancer include, but are not limited to, a genetic mutation associated with development of cancer; a genetic polymorphism associated with development of cancer; increased and/or decreased expression and/or activity of a protein associated with cancer; habits and/or lifestyles associated with development of cancer, including smoking, a sedentary lifestyle, and a high-fat diet; a family history of the cancer; and/or exposure to certain chemicals. Exemplary specific risk factors for developing one or more cancers include: exposure to asbestos, exposure to formaldehyde, exposure to acrylamide, chronic exposure to artificial sweeteners including saccharine, the presence of specific mutations in the BRCA1 and/or BRCA2 gene, exposure to diethylstilbestrol (DES), and prolonged exposure to direct sunlight.

Anti-Cancer Agents

In some embodiments, provided methods further include administering to the subject one or more of an anticancer agent and ionizing radiation. It is contemplated that Treg ablating agents or functional equivalents, analogs or derivatives thereof may be used in combination with any anti-cancer agent.

Exemplary traditional therapies or anticancer agents include, without limitation: surgery, radiotherapy (e.g., γ-radiation, neutron beam radiotherapy, electron beam radiotherapy, proton therapy, brachytherapy, and systemic radioactive isotopes, to name a few), endocrine therapy, biologic response modifiers (e.g., interferons, interleukins, and tumor necrosis factor (TNF) to name a few), hyperthermia and cryotherapy, agents to attenuate any adverse effects (e.g., antiemetics), alkylating drugs (e.g., mechlorethamine, chlorambucil, cyclophosphamide, melphalan, ifosfamide), antimetabolites (e.g., methotrexate), purine antagonists and pyrimidine antagonists (e.g., 6-mercaptopurine, 5-fluorouracil, cytarabile, gemcitabine), spindle poisons (e.g., vinblastine, vincristine, vinorelbine, paclitaxel), podophyllotoxins (e.g., etoposide, irinotecan, topotecan), antibiotics (e.g., doxorubicin, bleomycin, mitomycin), nitrosoureas (e.g., carmustine, lomustine), inorganic ions (e.g., cisplatin, carboplatin), enzymes (e.g., asparaginase), and hormones (e.g., tamoxifen, leuprolide, flutamide, and megestrol), to name a few. Additional non-limiting examples include anastrozole, letrozole, erlotinib, iressa, tarceva, doxorubicin, cyclophosphamide, gemcitabine, adriamycin, and trastuzumab and/or any other approved chemotherapeutic drugs. Any and all of these therapies may be used in connection with some embodiments of the present invention.

In some embodiments, anti-cancer agents include any treatment comprising administering an immunomodulator to an individual, wherein an immunomodulator induces, enhances, or suppresses the immune response. In some embodiments, immunomodulators comprise, for example, granulocyte colony-stimulating factor (G-CSF), interferons, cellular membrane fractions from bacteria, IL-2, IL-7, IL-12, various chemokines, synthetic cytosine phosphate-guanosine (CpG), oligodeoxynucleotides and glucans. In some embodiments, an anti-cancer agent is an anti-CTLA4 agent, an anti-PD-1 agent, and/or an anti-PD-L1 agent. In some embodiments, an anti-cancer agent is ionizing radiation.

Various anti-cancer agents, in particular, cancer immunotherapies, are available and may be used in accordance with various embodiments. Generally, cancer immunotherapies tend to induce an immune response. YERVOY® is an example recently approved by the Food and Drug Administration for the treatment of advanced melanoma. YERVOY® is a human anti-CTLA-4 antibody that is thought to induce the immune response by blocking activity of the T cell inhibitor CTLA-4. ONCOPHAGE® is an example in use in Russia for the treatment of renal carcinoma. ONCOPHAGE® is a vaccine that stimulates a cancer-cell specific immune response by introducing cancer cell antigens, including the gp96 heat shock protein.

In some embodiments, suitable immunotherapies are cell-based immunotherapies. Cell-based immunotherapies are generally based on the principal that the immune system can be programmed to attack cancer cells by specifically introducing to it an antigen specific for or more prevalent on cancer cells.

Dendritic cells, a type of antigen presenting cell, are one target for cell-based immunotherapy. Typically, in dendritic cell-based immunotherapy, dendritic cells are harvested from a patient. These cells are then either pulsed with an antigen or transfected with a viral vector. Upon transfusion back into the patient these activated cells present the tumor antigen to effector lymphocytes (CD4+ T cells, CD8+ T cells, and B cells). This initiates a cytotoxic response against cells expressing tumor antigens. The Dendreon cancer vaccine PROVENGE® is one example of this approach. With the PROVENGE® therapeutic cancer vaccine, a patient's own dendritic cells are isolated and treated with factors to induce activation in conjunction with the antigen prostatic acid phosphatase, a phosphatase present in 95% of prostate cancer cells. Once the dendritic cells are returned to the patient, they activate T-cells specific to prostatic acid phosphatase and the T-cells prostate cancer cells expressing the phosphatase. The precise mechanism of this action, however, has not been fully established. Other vaccines (e.g., cancer vaccines) are available in the art and can be used to practice the present invention.

In some embodiments, provided methods and compositions include administration of ionizing radiation. Generally, the term “ionizing radiation” includes radiation composed of particles that individually carry enough kinetic energy to liberate an electron from an atom or molecule, ionizing it. Ionizing radiation includes both subatomic particles moving at relativistic speeds and electromagnetic waves. Common particles include alpha particles, beta particles, neutrons, and various other particles such as mesons. Electromagnetic waves such as gamma rays, x-rays, and upper vacuum ultraviolet wavelength waves may be appropriate ionizing radiation according to some embodiments.

Typically, ionizing or other radiation is quantified according to gray units. The gray (Gy) is the SI derived unit of absorbed dose, specific energy, and kerma and is defined as the absorption of one joule of such energy by one kilogram of matter, typically water.

The amount of ionizing radiation administered according to any particular embodiment may vary according to the particular clinical presentation of a subject. It is contemplated that the appropriate dose of ionizing radiation will be determined in accordance with sound medical judgment. In some embodiments, the amount of ionizing radiation administered is between 1 Gy and about 1,000 Gy, about 5 Gy and about 900 Gy, about 10 Gy to about 800 Gy, about 10 Gy to about 700 Gy, about 10 Gy to about 600 Gy, about 10 Gy to about 500 Gy, about 10 Gy to about 400 Gy, about 10 Gy to about 300 Gy, about 10 Gy to about 200 Gy, about 10 Gy to about 100 Gy, about 5 Gy and about 15 Gy, between about 7.5 Gy and about 12 Gy, or between about 10 Gy and about 12 Gy. In some embodiments, the amount of ionizing radiation administered is about 12 Gy. In some embodiments, the amount of ionizing radiation is greater than about 10, 20, 30, 40, 50, 60, 70, 80, 90, 100, 200, 300, 400, 500, or 1,000 Gy. In some embodiments, the amount of ionizing radiation is less than about 1,000, 900, 800, 700, 600, 500, 400, 300, 200, 100, 90, 80, 70, 60, or 50 Gy.

Pharmaceutical Compositions

In some embodiments, the present invention provides pharmaceutical compositions comprising one or more provided Treg ablating agent together with one or more pharmaceutically acceptable excipients.

In some embodiments, provided pharmaceutical compositions may be prepared by any appropriate method, for example as known or hereafter developed in the art of pharmacology. In general, such preparatory methods include the step of bringing a provided Treg ablating agent into association with one or more pharmaceutically acceptable excipients, and then, if necessary and/or desirable, shaping and/or packaging the product into an appropriate form for administration, for example as or in a single- or multi-dose unit.

In some embodiments, compositions may be prepared, packaged, and/or sold in bulk, as a single unit dose, and/or as a plurality of single unit doses. As used herein, a “unit dose” is a discrete amount of the pharmaceutical composition comprising a predetermined amount of one or more provided Treg ablating agent. The amount of the provided Treg ablating agent is generally equal to the dosage of the provided Treg ablating agent which would be administered to a subject and/or a convenient fraction of such a dosage such as, for example, one-half or one-third of such a dosage.

In many embodiments, provided pharmaceutical compositions are specifically formulated for mucosal delivery (e.g., oral, nasal, rectal or sublingual delivery).

In some embodiments, appropriate excipients for use in provided pharmaceutical compositions may, for example, include one or more pharmaceutically acceptable solvents, dispersion media, granulating media, diluents, or other liquid vehicles, dispersion or suspension aids, surface active agents and/or emulsifiers, isotonic agents, thickening or emulsifying agents, preservatives, solid binders, lubricants, disintegrating agents, binding agents, preservatives, buffering agents and the like, as suited to the particular dosage form desired. Alternatively or additionally, pharmaceutically acceptable excipients such as cocoa butter and/or suppository waxes, coloring agents, coating agents, sweetening, flavoring, and/or perfuming agents can be utilized. Remington's The Science and Practice of Pharmacy, 21^{st} Edition, A. R. Gennaro (Lippincott, Williams & Wilkins, Baltimore, Md., 2005; incorporated herein by reference) discloses various excipients used in formulating pharmaceutical compositions and known techniques for the preparation thereof.

In some embodiments, an appropriate excipient is at least 95%, at least 96%, at least 97%, at least 98%, at least 99%, or 100% pure. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or other International Pharmacopoeia.

In some embodiments, liquid dosage forms (e.g., for oral and/or parenteral administration) include, but are not limited to, emulsions, microemulsions, solutions, suspensions, syrups, and/or elixirs. In addition to provided Treg ablating agent(s), liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such a CREMOPHOR®, alcohols, oils, modified oils, glycols, polysorbates, cyclodextrins, polymers, and/or combinations thereof.

In some embodiments, injectable preparations, for example, sterile aqueous or oleaginous suspensions, may be formulated according to known methods using suitable dispersing agents, wetting agents, and/or suspending agents. Sterile liquid preparations may be, for example, solutions, suspensions, and/or emulsions in nontoxic parenterally acceptable diluents and/or solvents, for example, as a solution in 1,3-butanediol. Among the acceptable vehicles and solvents that may be employed, for example, are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of liquid formulations.

Liquid formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, one or more strategies may be utilized prolong and/or delay the effect of a provided Treg ablating agent after delivery.

In some embodiments, provided pharmaceutical compositions may be formulated as suppositories, for example for rectal or vaginal delivery. In some embodiments, suppository formulations can be prepared by mixing utilizing suitable non-irritating excipients such as cocoa butter, polyethylene glycol or a suppository wax which are solid at ambient temperature but liquid at body temperature and therefore melt in the body (e.g., in the rectum or vaginal cavity) and release the provided Treg ablating agent.

In some embodiments, solid dosage forms (e.g., for oral administration) include capsules, tablets, pills, powders, and/or granules. In such solid dosage forms, the provided Treg ablating agent(s) may be mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

In some embodiments, solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

Exemplary enteric coatings include, but are not limited to, one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); HP55; polyvinyl acetate phthalate (PVAP); methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophtalate; acrylic resin; shellac, and combinations thereof.

In some embodiments, solid dosage forms may optionally comprise opacifying agents and can be of a composition that they release the provided Treg ablating agent(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In some embodiments, the present invention provides compositions for topical and/or transdermal delivery, e.g., as a cream, liniment, ointment, oil, foam, spray, lotion, liquid, powder, thickening lotion, or gel. Particular exemplary such formulations may be prepared, for example, as products such as skin softeners, nutritional lotion type emulsions, cleansing lotions, cleansing creams, skin milks, emollient lotions, massage creams, emollient creams, make-up bases, lipsticks, facial packs or facial gels, cleaner formulations such as shampoos, rinses, body cleansers, hair-tonics, or soaps, or dermatological compositions such as lotions, ointments, gels, creams, liniments, patches, deodorants, or sprays.

In some embodiments, provided compositions are stable for extended periods of time, such as 1 week, 2 weeks, 1 month, 2 months, 6 months, 1 year, 2 years, 3 years, or more. In some embodiments, provided compositions are easily transportable and may even be sent via traditional courier or other package delivery service. Accordingly, some embodiments may be useful in situations of disease outbreak, such as epidemics, or attacks with biological agents at least in part due to their ability to be stored for long periods of time and transported quickly, easily, and safely. Such attributes may allow for rapid distribution of provided compositions to those in need.

In some embodiments, it may be advantageous to release Treg ablating agent(s), for example, a CCR4 antibody, at various locations along a subject's gastrointestinal (GI) tract. In some embodiments, it may be advantageous to release Treg ablating agent(s), for example, an antigen, in a subject's mouth as well as one or more locations along the subject's GI tract. Accordingly, in some embodiments, a plurality of provided compositions (e.g., two or more) may be administered to a single subject to facilitate release of Treg ablating agent(s) at multiple locations. In some embodiments, each of the plurality of compositions has a different release profile, such as provided by various enteric coatings, for example. In some embodiments, each of the plurality of compositions has a similar release profile. In some embodiments, the plurality of compositions comprises one or more Treg ablating agents. In some embodiments, each of the plurality of administered compositions comprises a different Treg ablating agent. In some embodiments, each of the plurality of compositions comprises the same Treg ablating agent.

Dosing

It is contemplated that a variety of dosing regimen may be used in accordance with various embodiments. In some embodiments, the step of ablating comprises administering at least two doses of a Treg ablating agent, separated by a period of time. In some embodiments, the step of ablating comprises administering at least three, four, five, six or more than six doses of a Treg ablating agent, each separated by a period of time. In some embodiments, the period of time between each administration is the same. In some embodiments, the period of time between each administration is different. In some embodiments, the period of time between doses may be 1 minute, 15 minutes, 30 minutes, 1 hour, 2 hours, 3 hours, 6 hours, 12 hours, 24 hours, 48 hours, 72 hours, 96 hours, 1 week, 2 weeks, or 1 month. In some embodiments, the period of time between doses is greater than 1 month. In some embodiments, each dose is administered substantially simultaneously (e.g., sequentially).

According to various embodiments comprising administration of two or more doses of a Treg ablating agent, the dose of Treg ablating agent may vary according to sound medical judgment. In some embodiments, each dose of a Treg ablating agent is the same. In some embodiments, each dose of a Treg ablating agent may vary from one or more other doses.

In some embodiments, a Treg ablating agent is administered at a dose equal to or approximating a therapeutically effective amount. In some embodiments, a therapeutically effective amount of a Treg ablating agent may be an amount ranging from about 0.001 to about 1,000 mg/kg. In some embodiments, a therapeutically effective amount may be, for example, about 0.001 to 500 mg/kg weight, e.g., from about 0.001 to 400 mg/kg weight, from about 0.001 to 300 mg/kg weight, from about 0.001 to 200 mg/kg weight, from about 0.001 to 100 mg/kg weight, from about 0.001 to 90 mg/kg weight, from about 0.001 to 80 mg/kg weight, from about 0.001 to 70 mg/kg weight, from about 0.001 to 60 mg/kg weight, from about 0.001 to 50 mg/kg weight, from about 0.001 to 40 mg/kg weight, from about 0.001 to 30 mg/kg weight, from about 0.001 to 25 mg/kg weight, from about 0.001 to 20 mg/kg weight, from about 0.001 to 15 mg/kg weight, from about 0.001 to 10 mg/kg weight. In some embodiments, the therapeutically effective amount described herein is provided in one dose. In some embodiments, the therapeutically effective amount described herein is provided in one day.

In some embodiments, a therapeutically effective dosage amount may be, for example, about 0.0001 to about 0.1 mg/kg weight, e.g. from about 0.0001 to 0.09 mg/kg weight, from about 0.0001 to 0.08 mg/kg weight, from about 0.0001 to 0.07 mg/kg weight, from about 0.0001 to 0.06 mg/kg weight, from about 0.0001 to 0.05 mg/kg weight, from about 0.0001 to about 0.04 mg/kg weight, from about 0.0001 to 0.03 mg/kg weight, from about 0.0001 to 0.02 mg/kg weight, from about 0.0001 to 0.019 mg/kg weight, from about 0.0001 to 0.018 mg/kg weight, from about 0.0001 to 0.017 mg/kg weight, from about 0.0001 to 0.016 mg/kg weight, from about 0.0001 to 0.015 mg/kg weight, from about 0.0001 to 0.014 mg/kg weight, from about 0.0001 to 0.013 mg/kg weight, from about 0.0001 to 0.012 mg/kg weight, from about 0.0001 to 0.011 mg/kg weight, from about 0.0001 to 0.01 mg/kg weight, from about 0.0001 to 0.009 mg/kg weight, from about 0.0001 to 0.008 mg/kg weight, from about 0.0001 to 0.007 mg/kg weight, from about 0.0001 to 0.006 mg/kg weight, from about 0.0001 to 0.005 mg/kg weight, from about 0.0001 to 0.004 mg/kg weight, from about 0.0001 to 0.003 mg/kg weight, from about 0.0001 to 0.002 mg/kg weight. The effective dose for a particular individual can be varied (e.g., increased or decreased) over time, depending on the needs of the individual.

Routes of Administration

In some embodiments, provided Treg ablating agents and compositions comprising the same may be formulated for any appropriate route of delivery. In some embodiments, provided Treg ablating agents and compositions comprising the same may be formulated for any route of delivery, including, but not limited to, bronchial instillation, and/or inhalation; buccal, enteral, interdermal, intra-arterial (IA), intradermal, intragastric (IG), intramedullary, intramuscular (IM), intranasal, intraperitoneal (IP), intrathecal, intratracheal instillation (by), intravenous (IV), intraventricular, mucosal, nasal spray, and/or aerosol, oral (PO), as an oral spray, rectal (PR), subcutaneous (SQ), sublingual; topical and/or transdermal (e.g., by lotions, creams, liniments, ointments, powders, gels, drops, etc.), transdermal, vaginal, vitreal, and/or through a portal vein catheter; and/or combinations thereof. In some embodiments, the present invention provides methods of administration of Treg ablating agents and compositions comprising the same via mucosal administration. In some embodiments, the present invention provides methods of administration of Treg ablating agents and compositions comprising the same via oral administration.

Kits

In some embodiments, the present invention further provides kits or other articles of manufacture which contain one or more Treg ablating agents or formulations containing the same, and provides instructions for its reconstitution (if lyophilized) and/or use. In some embodiments, a kit may comprise (i) at least one provided Treg ablating agent or composition comprising the same; and (ii) at least one pharmaceutically acceptable excipient; and, optionally, (iii) instructions for use.

Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container may holds formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, a container may contain a single dose of a stable formulation containing one or more Treg ablating agents. In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/ml (e.g., at least 5 mg/ml, at least 10 mg/ml, at least 20 mg/ml, at least 30 mg/ml, at least 40 mg/ml, at least 50 mg/ml, at least 75 mg/ml, at least 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

In some embodiments, kits include multiple (e.g., 1, 2, 3, 4, 5, 6, 7, 8, 9, 10, 11, 12, 13, 14, 15, 16, 17, 18, 19, 20, or more) doses of provided Treg ablating agents and/or compositions comprising the same. In some embodiments, kits include multiple (e.g., 1, 2, 3, 4, 5, 6, 7, 8, 9, 10, 11, 12, 13, 14, 15, 16, 17, 18, 19, 20, or more) populations of provided Treg ablating agents and/or compositions comprising the same having different functional elements (e.g., Treg ablating agents). In some embodiments, multiple populations of provided Treg ablating agents and/or compositions comprising the same are packaged separately from one another in provided kits. In some embodiments, provided kits may include provided compositions and one or more other therapeutic agents intended for administration with the provided compositions.

### EXAMPLES

### Example 1

### Materials and Methods

Unless otherwise specified, the methods used in Examples 2-8 are as follows:

Mice

Foxp3^{DTR} were generated in the laboratory and previously described (Kim et al., 2007). β2M^{-/-} mice were purchased from Taconic. Mice bearing the MMTV-rtTA and TetO-PyMT:IRES:Luc transgenes were generously provided by Dr. H. Varmus. C57BL/6 MMTV-PyMT mice were a kind gift from Dr. M. O. Li. All animal studies were performed in accordance with an IACUC-approved protocol at the Memorial Sloan-Kettering Cancer Center.

Animal Experiments

For oncogene induction, mice were placed on doxycycline-impregnated food pellets (625 ppm; Harlan-Teklad). For regulatory T-cell (Treg) cell ablation studies, diptheria toxin (Sigma-Aldrich) was injected intravenously at 50 μg or 25 μg per kg of body weight at indicated times. Mammary tumorigenic cell lines were generated via enzymatic dissociation of invasive tumors from MMTV-PyMT mice, briefly expanded in Dulbecco's modified Eagle's, high glucose medium supplemented with 10% FBS and transduced with a Firefly Luciferase retroviral vector using standard techniques. For orthotopic implantation studies, 100,000 cells were resuspended in PBS and mixed in a 1:1 ratio with growth factor-reduced Matrigel (BD), and injected in the mammary fat pad of isofluorine-anesthetized mice. Primary tumor outgrowth was monitored daily by taking measurements of the tumor length (L) and width (W). Tumor volume was calculated as ΠLW²/6.

For experimental lung metastasis assays, 500,000 cells were resuspended in PBS and inoculated via tail vein injection. Lung metastatic burden was quantified by counting the number of metastatic nodules under a dissection scope (Olympus), ex-vivo bioluminescence using an IVIS200 imager (Xenogen), or calculating the ratio between the area covered by metastasis over the total area of the lung in histological sections. CTLA-4 (clone 9D9), PD-1 (clone RPM1-14), and PD-L1 (clone 10F.9G2) antibodies were administered intraperitoneally at days 0, 3 and 6 at a dose of 100 μg, 250 μg and 100 μg per mouse, respectively, as indicated in the text. IFN-γ (clone XMG1.2), NK cells (clone PK136) and CD8 T cells (clone 2.43) depletion was achieved through i.p. injection of 1 mg, 300 μg and 250 μg respectively, together with the second dose of human diphtheria toxin (DT) (IFN-γ and NK) or 4 days after DT injection (CD8). All antibodies for animal studies were obtained from BioXcell. Radiation was administered in a single dose of 12 Gy when tumors reached approximately 100 mm³ or 250 mm³ of volume using a X-RAD 225Cx microirradiator. Briefly, individual mice were anesthetized using isofluorine, and positioned on a platform where a cone-beam CT imaging of the animal was done to allow targeting the radiation field to the tumor, avoiding normal structures.

Cytokine Array

Cytokines and chemokines were measured using a multiplex Luminex bead assay (Millipore). Tumors were lysed in buffer containing 50 mM Tris, 150 mM NaCl, 1% NP-40, 1 mM EDTA and protease inhibitors. Cleared lysates were quantified and extracts bearing 20 μg of total protein were incubated with mouse cytokine/chemokine magnetic bead panels I, II, and III from Milliplex, following manufacturer's instructions.

Histology

For histological analyses, tissues were fixed in 10% neutral buffered formalin and routinely processed for hematoxylin and eosin staining Apoptosis (cleaved caspase 3), proliferation (Ki67), leukocyte (CD45) and macrophage (IBA1) stainings were performed using automated IHC techniques by the Molecular Cytology Core Facility, and quantified using Metamorph analysis.

Flow Cytometry

Tumor infiltrating lymphocytes were isolated by enzymatic dissociation of tumors using LiberaseTL (Roche), digested for 25 minutes followed by Percoll (VWR) centrifugation to eliminate dead cells. Intracellular Foxp3 staining was performed using Foxp3 mouse Treg cell staining kit (eBioscience). Cytokine staining was performed after stimulation of splenocytes or isolated TILs with PMA (50 ng/ml) and Ionomycin (500 ng/ml) for 4-5 hr. in the presence of Golgi-Plug (BD Biosciences). All antibodies used for flow cytometry staining were purchased from eBioscience or BD Biosciences. Stained cells were analyzed in a LSRII flow cytometer (BD Biosciences). Data were analyzed using FlowJo software (TreeStar).

FACS Isolation and Quantitative PCR Analysis

For qPCR analysis, tumors were processed by enzymatic digestion as previously described and myeloid or T cells were sorted based on their surface expression of CD45, TCRβ, CD11B, and Gr1 using a FACS Aria2 (BD). Sorted cells were lysed in Trizol reagent (Invitrogen), and reverse-transcribed using SuperScript III Reverse Transcriptase (Invitrogen). Semi-quantitative PCR was performed using the following SybrGreen primers:

Beta-actin:
forward
(SEQ ID NO: 1)
5′-CTAAGGCCAACCGTGAAAAG-3′;
reverse
(SEQ ID NO: 2)
5′-ACCAGAGGCATACAGGGACA-3′;
IFN-γ:
forward
(SEQ ID NO: 3)
5′-ATCTGGAGGAACTGGCAAAA-3′;
reverse
(SEQ ID NO: 4)
5′-TTCAAGACTTCAAAGAGTCTGAGGTA-3′;
CXCL9:
forward
(SEQ ID NO: 5)
5′-TTTTCCTTTTGGGCATCATCTT-3′;
reverse
(SEQ ID NO: 6)
5′-AGCATCGTGCATTCCTTATCACT-3′;
CXCL10:
forward
(SEQ ID NO: 7)
5′-GAAATCATCCCTGCGAGCCT-3′;
reverse
(SEQ ID NO: 8)
5′-TTGATGGTCTTAGATTCCGGATTC-3′.

Statistical Analysis

All statistical analysis was performed using Student's t-test or ANOVA analysis as indicated with the Prism software (GraphPad).

### Example 2

### Therapeutic Regulatory T-Cell Ablation Affects the Growth of Large Mammary Tumors and Established Lung Metastasis

Carcinoma cells isolated from C57BL/6 mice expressing a transgene encoding the PyMT oncogene under control of the MMTV promoter were implanted in a knock-in mouse generated where the Foxp3 locus controls expression of the human diphtheria toxin (DT) receptor (Foxp3^{DTR}). Orthotopic implantation of only 1×10⁵ tumor cells in the inguinal mammary gland of virgin female Foxp3^{DTR} mice on a C57BL/6 background results in uniformly growing mammary tumors that metastasize to the lungs with complete penetrance in approximately 3 to 4 weeks. This strategy was used to evaluate tumor growth in large cohorts of mice with synchronous, rapidly progressing metastatic mammary tumors.

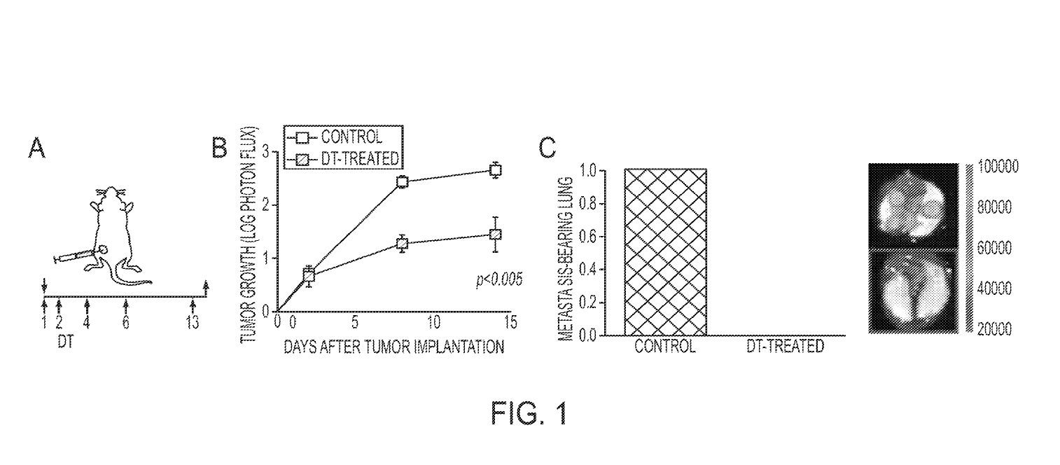

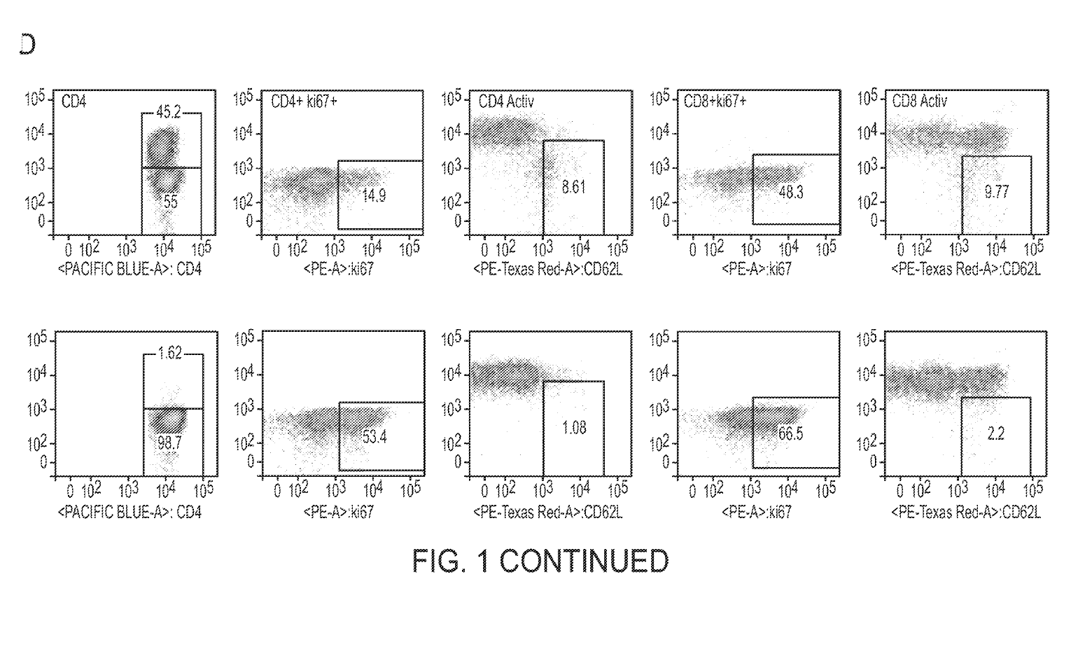

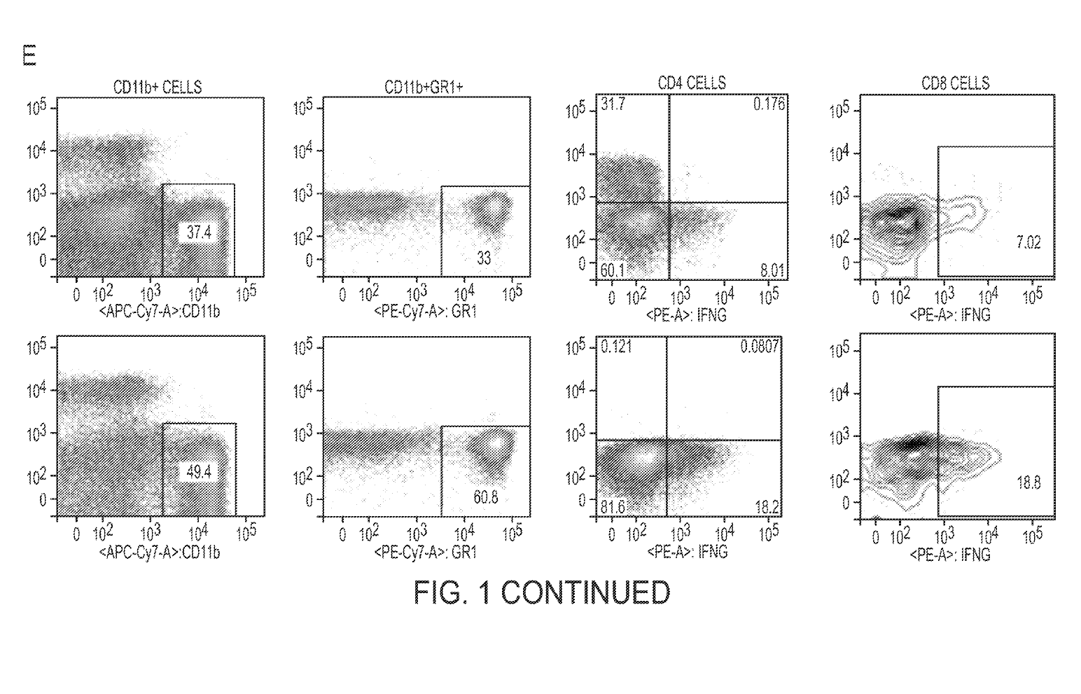

Foxp3+ Treg cells were ablated through injection of 50 m/kg of DT at days 1, 2, 4, 6 and 13 after tumor cell implantation (FIG. 1A); significant reduction in tumor growth and incidence of lung metastasis was observed (FIG. 1B-C). Flow cytometric analysis of lymphocyte populations isolated from enzymatically-dissociated tumors demonstrated that the extent of Treg cell ablation was greater than 99% (FIG. 1D). Expansion and activation of CD4+ and CD8+ T cell subsets was observed based on the increased expression of Ki67 and CD44 and decreased levels of CD62L (FIG. 1D). In addition, the proportion of CD4+ and CD8+ T cells expressing IFN-γ and TNFα was markedly augmented (FIG. 1E and data not shown). Furthermore, we also observed an increase in immature myeloid cells (FIG. 1E).

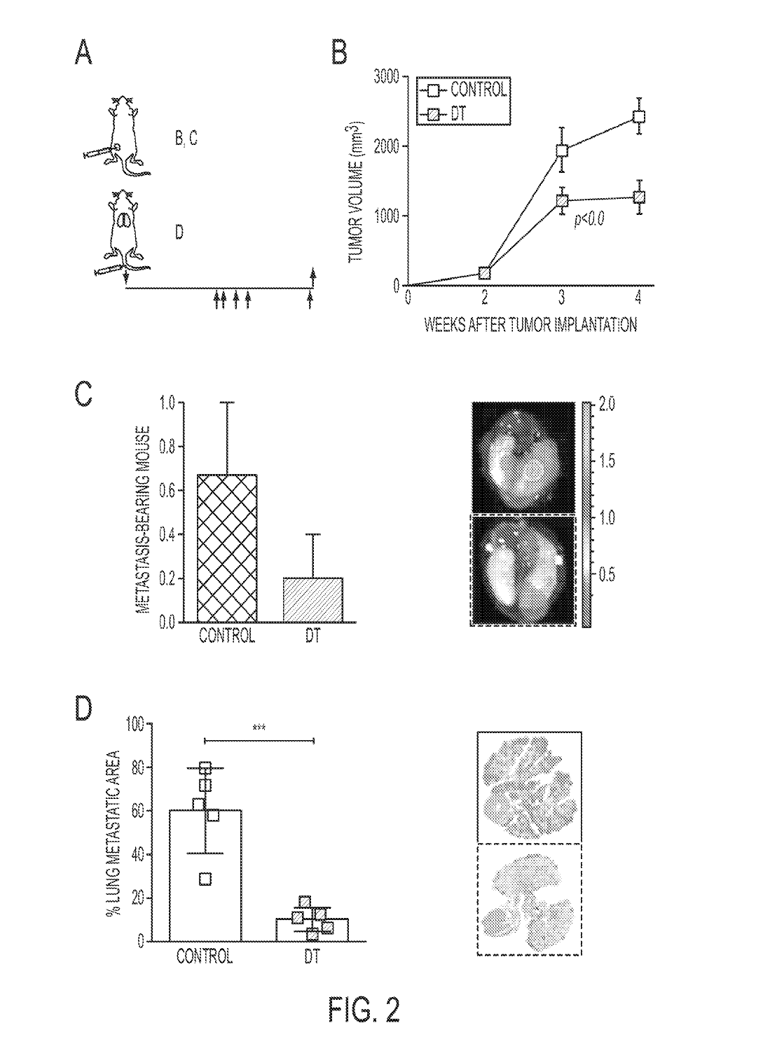

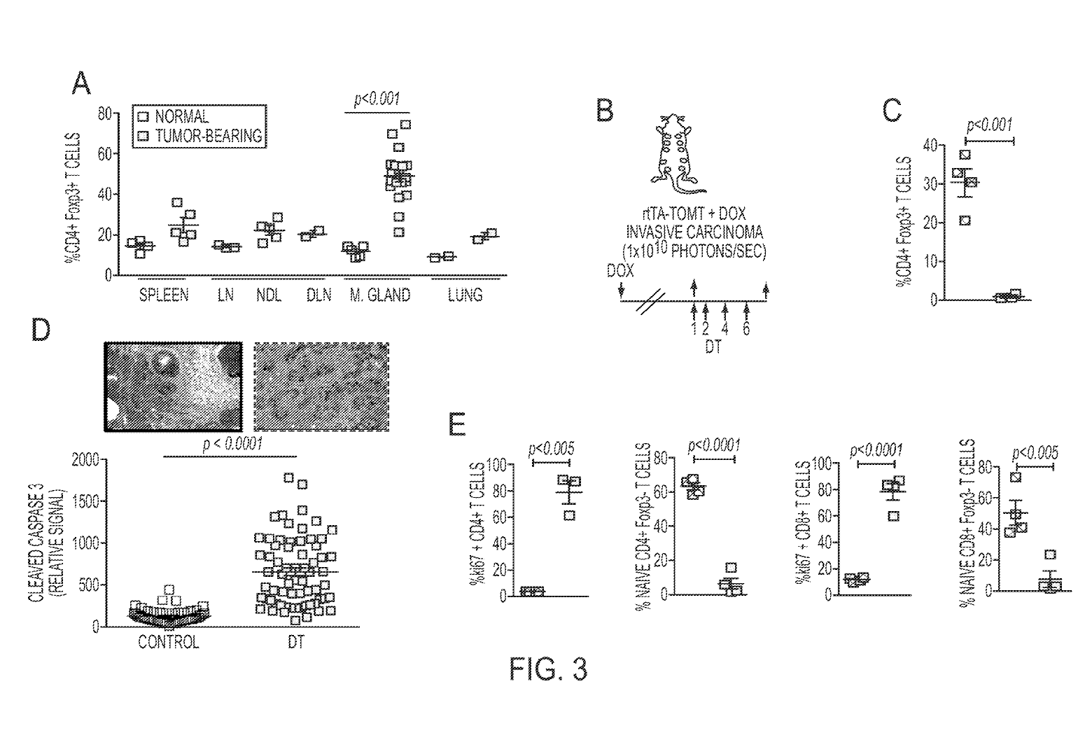

In an effort to assess the effect of Treg ablation in established tumors that reached exponential growth (approximately 250 mm³) human diphtheria toxin (DT) was administered to Foxp3^{DTR} mice. As shown in FIG. 2A, DT treatment of Foxp3^{DTR} mice with large tumors resulted in significant reduction of tumor burden (FIG. 2A-B). In addition, the incidence and size of lung metastasis in mice bearing large tumors was significantly reduced upon depletion of Treg cells (FIG. 3C). Without wishing to be held to a particular theory, the observed reduction in lung metastatic burden may be secondary to reduced primary tumor volume in DT-treated animals, since metastatic load is thought to be proportional to primary tumor size (see Heimann and Hellman, 2000; Minn et al., 2007).

To investigate whether the beneficial effect of Treg ablation on lung metastasis in was independent of the diminished primary tumor growth, animals were treated with DT following establishment of lung metastasis, two weeks after tail vein inoculation of 5×10⁵ PyMT cells (FIG. 3A). Analysis performed 2 weeks after DT injection showed that tumor burden in the lungs was markedly reduced as determined by histological quantification of tumor areas in lung sections, demonstrating a pronounced direct effect of Treg cell ablation on the disseminated tumors (FIG. 3D). This Example demonstrates that Treg cell ablation is therapeutic not only for newly formed, but also large, rapidly growing primary mammary tumors and fully established lung metastasis.

### Example 3

### Regulatory T-Cell Ablation Results in Tumor Cell Death in Spontaneously Developing Oncogene-Driven Mammary Tumors

To determine whether the potent restraint of cancer progression and metastasis in the orthotopic transplantation model of breast carcinogenesis could be applied to a genetically induced oncogene-driven tumors, the Foxp3^{DTR} allele was introduced into mice co-expressing PyMT oncogene and a luciferase reporter under a doxycyclineinducible promoter, and reverse tetracycline-controlled transactivator under the MMTV promoter (MMTV-rtTA; tet-O-MT:IRES:Luc or TOMT) (see Podsypanina et al., 2008 for a description of an example of such a construct). Upon doxycycline administration, these mice developed tumors in all mammary glands.

Analysis of tumor-infiltrating lymphocytes showed that Treg cells were highly enriched within the CD4⁺ T cell subset (FIG. 3A). Mice were allowed to develop large invasive carcinomas that reached a photon flux of 1×10¹⁰ photons per second. Analysis was performed 10 days after the initial dose of DT (FIG. 3B). At that time the mice were fully active and did not present any signs of morbidity despite sustained Treg cell ablation during the time frame of the experiment (FIG. 3C). Because asynchronous and slow tumor growth in TOMT mice precludes the accurate evaluation of the effect of Treg ablation on growth kinetics, the expression of cleaved caspase-3 in cancer cells, a marker of apoptotic death, was assessed as a means to assess the consequence of Treg cell ablation on tumor viability. A significant increase in apoptosis of tumor cells in mice treated with DT compared to control mice injected with PBS was observed (see FIG. 3D). Concomitantly, a significant expansion and activation of CD4⁺ and CD8⁺ T cells in tumors was observed in Treg cell-depleted mice (see FIG. 3E).

This Example indicates that Treg cells represent a major cellular mechanism facilitating tumor progression by maintaining viability in this experimental model of oncogene-driven breast cancer.

### Example 4

### Transient Regulatory T-Cell Ablation is Sufficient to Achieve Significant Reduction in Tumor Burden

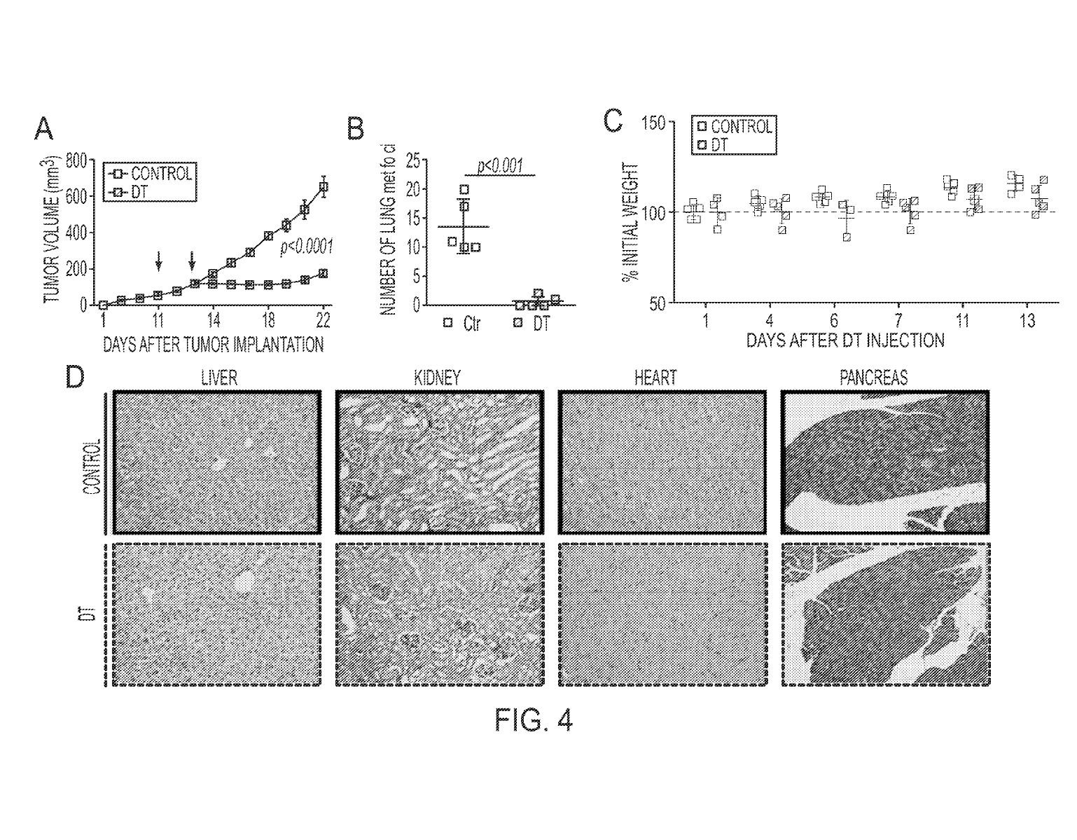

To minimize the potential side effects of Treg ablation and test whether continuous ablation was required to achieve the observed reduction in orthotopic tumor growth, the dose and frequency of the DT administration was limited. Specifically, tumor-bearing animals were given only two 25 μg/kg doses of DT once tumors reached approximately 100 mm³. This treatment regimen allowed for efficient (>99%), yet transient Treg ablation with minimal morbidity (slight short-term weight loss with quick recovery; FIG. 4C) and no gross organ immunopathology evaluated by histological examination 2 weeks after DT (FIG. 4D).

Remarkably, despite lack of pronounced generalized immunopathology this brief ablation of Treg cells significantly hindered primary tumor growth (FIG. 4A), and resulted in the almost complete disappearance of metastatic tumor nodules in the lungs (FIG. 4B). This Example demonstrates that efficient ablation of Treg cells for a relatively short period of time may provide similar therapeutic benefit to persistent ablation, with a reduced chance of dangerous side effects.

### Example 5

### Regulatory T-Cell Ablation Promotes a Tumor-Suppressive Microenvironment

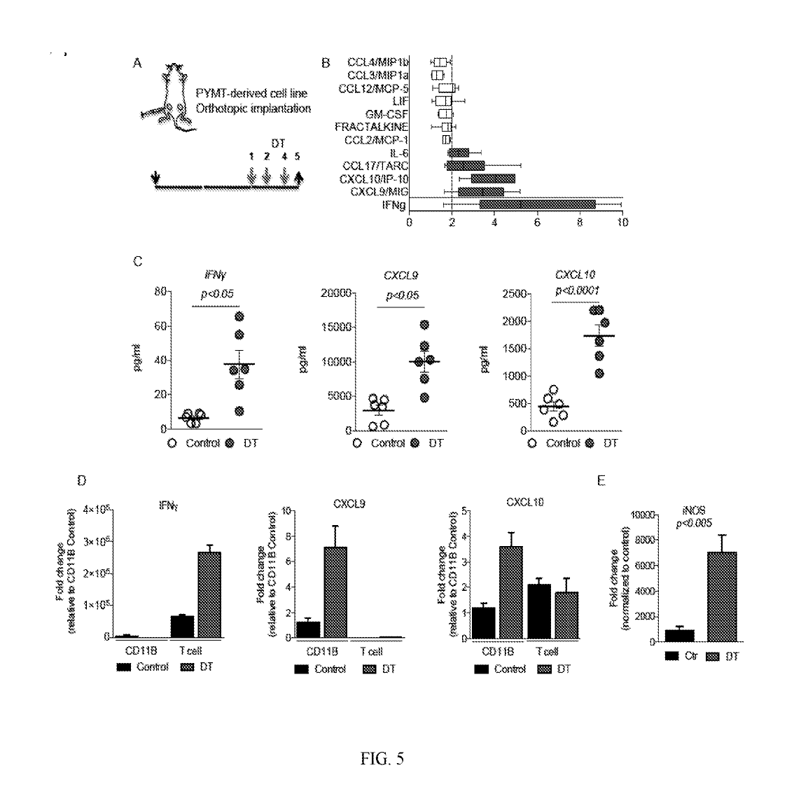

Without wishing to held to a particular theory, it is possible that Treg cells could be beneficial to cancer cell growth and tumor progression in at least two ways. One, Treg cells may suppress components of the adaptive immune system providing protection from tumor cell killing. Alternatively, Treg cells may modulate the microenvironment via soluble mediators that may directly or indirectly promote tumor progression. In order to better understand the early changes taking place in the tumor microenvironment upon Treg cell ablation, a protein array of 66 cytokines and chemokines on tumor lysates prepared on day 5 after DT administration was analyzed to evaluate early changes in these soluble mediators (FIG. 5A). Comparison of control and DT-treated lysates revealed significant increments in 12 cytokines, although only 5 of them increased above a 2-fold threshold (FIG. 5B). The most prominent change was observed in IFN-γ, a potent immune-modulator and anti-tumor cytokine, followed by CXCL9 and CXCL10 (FIG. 5C). These two chemokines are produced by several cell types in response to IFN-γ, and serve as chemoattractant for CXCR3-expressing leukocytes, most notably TH1 and NK cells, but also monocytes, endothelial cells and some epithelial cells.

To validate these observations and determine the source of each cytokine, T cells (CD45+CD3+CD11B−Gr1−) and myeloid cells (CD45+CD3−CD11B+Gr1−) cells were isolated from an independent group of control and DT-treated tumors by fluorescence activated cell sorting. Using primer-specific semi-quantitative PCR, the mRNA levels in these two populations was determined. As shown in FIG. 5D, IFN-γ mRNA was produced in the T cell compartment and increased significantly upon Treg ablation, whereas CXCL9 and CXCL10 mRNA was significantly increased in the myeloid compartment upon DT treatment, perhaps as a response to IFN-γ. Because IFN-γ is a potent classic activator of macrophages, we quantified the mRNA levels of iNOS—a prototypical IFN-γ induced M1 polarized macrophage effector—in the myeloid cell compartment, and observed a high fold induction of iNOS upon Treg ablation (FIG. 5E). Without wishing to be held to a particular theory, these results suggest that Treg ablation leads to a strong IFN-γ-mediated anti-tumor milieu that can stimulate TH1, NK and M1 responses against the tumor.

### Example 6

### Tumoricidal Effects are Mediated Via IFN-γ, but not CD8+ T-Cells or NK Cells

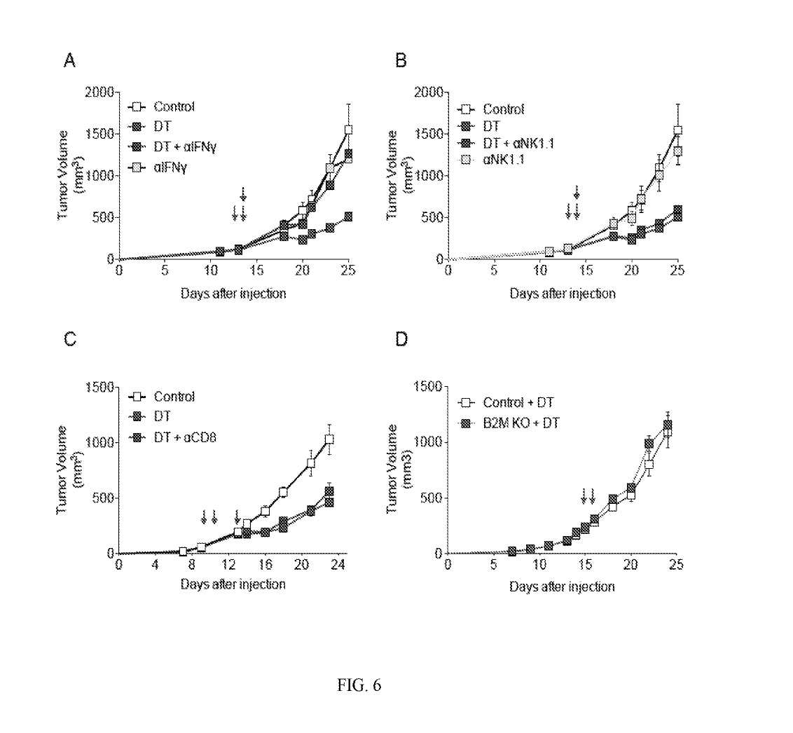

Given the predominance of IFN-γ in Treg-depleted tumors, its functional role in the observed delayed tumor progression was evaluated. To this end, mice were injected with 1 mg IFN-γ neutralizing antibody alone or in combination with DT. Although anti-IFN-γ antibody treatment alone did not have an impact on tumor growth in control mice, combination of anti-IFN-γ antibody and DT almost completely abolished the effect of Treg ablation on the kinetics of tumor growth (FIG. 6A).

In order to determine whether the IFN-γ effect was mediated through cytotoxic T or NK cells, Treg cells were ablated in the presence of NK- or CD8-depleting antibodies. NK cell depletion using NK1.1 antibody did not have a detectable effect on growth of control or Treg-depleted tumors (FIG. 6B). In addition, administration of a CD8 depleting antibody during the course of Treg ablation did not affect the tumor growth reduction caused by Treg ablation, nor the growth of control tumors (FIG. 6C).

To corroborate this finding, mice lacking β2-microglobulin, required for MHC class I expression and proper maturation of CD8⁺ T cells, were crossed with Foxp3^{DTR} exemplary methods may be found in Gasteiger et al., 2013). Treg cell ablation in control or DT-treated Foxp3^{DTR} β2M^{-/-} mice resulted in comparable determent of tumor progression and indistinguishable tumor growth profiles (FIG. 6D), in agreement with antibody-mediated depletion. In contrast, when CD4 cells were depleted from the DT-treated tumors, there was a pronounced attenuation in the Treg-mediated antitumor effect (data not shown).

Together, the results in this Example show that NK and CD8⁺ T cells are not necessary for the anti-tumor effect of Treg cell ablation, which is partially dependent on CD4⁺ T cells and requires IFN-γ. In addition, these observations suggest that NK and CD8⁺ T cells are dispensable as the source of IFN-γ.

### Example 7

### Checkpoint Blockade does not Improve Regulatory T-Cell Ablation Effect on Mammary Tumor Progression

In an effort to determine if the observed potent anti-tumor effect achieved via Treg cell ablation could be used in conjunction with currently known anti-tumor therapies, the use of checkpoint inhibitors in conjunction with Treg cell ablation was explored. Highly expressed on activated and chronically stimulated (“exhausted”) effector cells, CTLA-4, PD-1 and its ligand PD-L1 are also present in high amounts on Treg cells (see Pardoll, 2012), and their antibody-mediated inhibition have proven a viable immunotherapeutic strategy to treat solid tumors in recent pre-clinical studies and clinical trials. Therefore, immune checkpoint blockade could potentially promote the effector response of newly recruited T cells in addition to reversing the exhausted state of pre-existing tumor-infiltrating T cells.

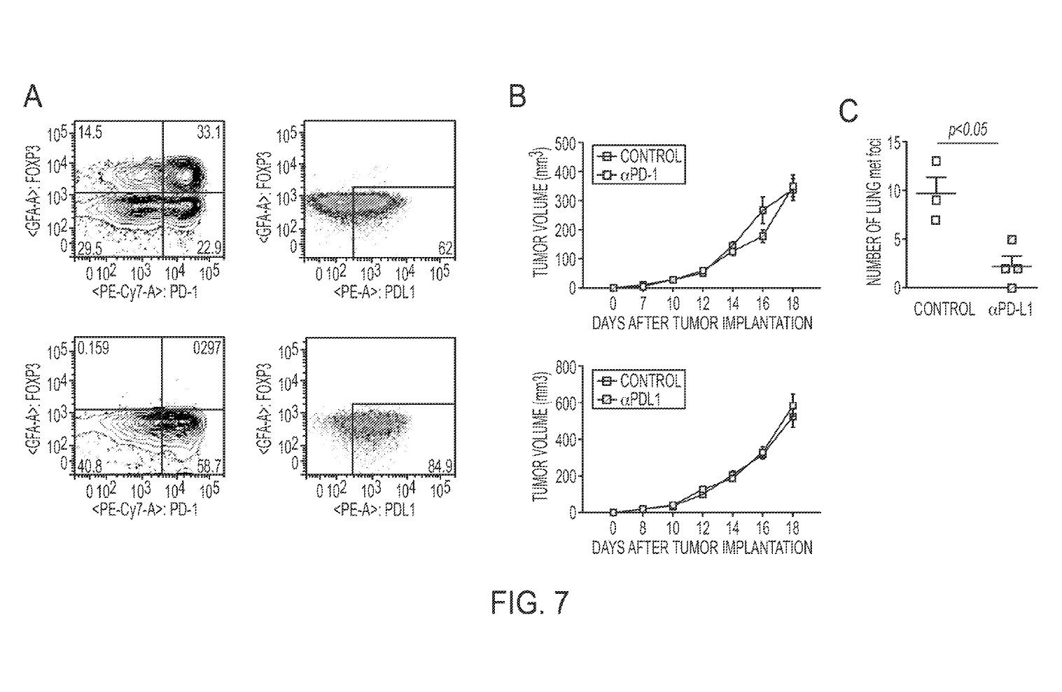

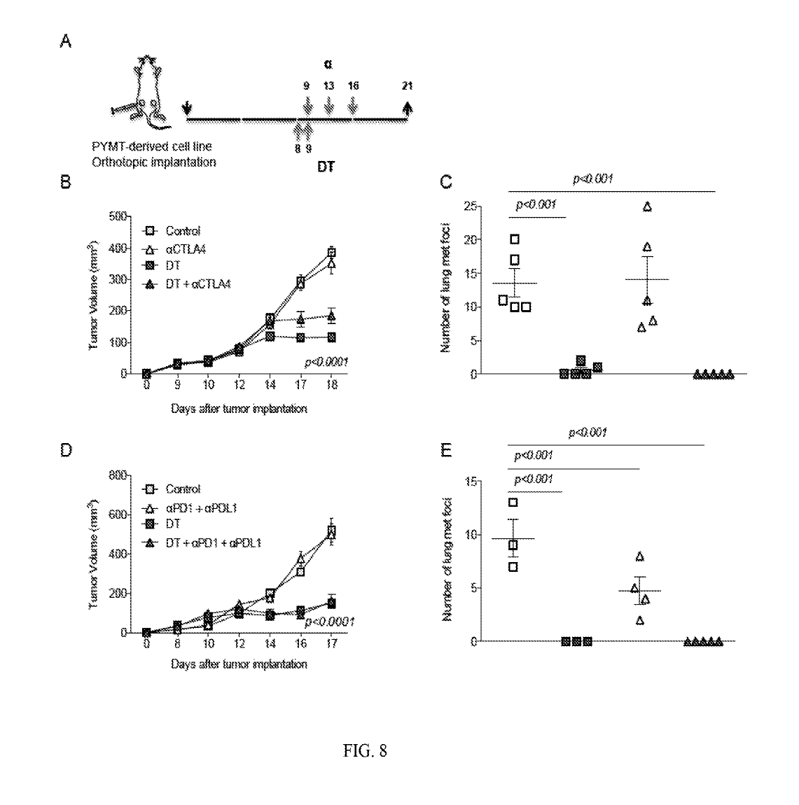

In support of this concept, tumor-infiltrating lymphocytes in Treg-depleted tumors exhibited a marked increase in the expression of the PD-1 receptor on effector T cells. Additionally, expression of the PD-L1 ligand was increased on both, T cells and myeloid cells (FIG. 7A). This study sought to explore whether, in combination with CTLA-4 or PD-1 checkpoint blockade, the therapeutic effect obtained through Treg ablation alone could be enhanced in the oncogene-driven orthotopic model of breast cancer used herein. First, the effects of targeting the CTLA-4 or PD-1/PD-L1 inhibitory pathways with blocking antibodies of corresponding specificity administered on days 0, 3 and 6 after tumors reached approximately 100 mm³ volume was analyzed. As shown in FIG. 8 A, B, D; FIG. 7B, blockade of either one of these pathways by CTLA-4 or PD-1 or PD-L1 or a combination of PD-1 and PD-L1 antibodies had no significant effect on the growth of PyMT-driven orthotopic tumors. It is of note that lung metastatic burden measured by enumerating tumor nodules on the lung surface was diminished by half upon the blockade of PD-1/PD-L1, but not CTLA-4 signaling (FIG. 8C-D; FIG. 7C).

A combination of DT with CTLA-4 antibody or with PD-1 and PD-L1 antibodies did not enhance the effect of Treg ablation alone on primary tumor progression (FIG. 2 B, D). Since DT treatment almost completely eliminated the appearance of metastatic nodules in the lungs, it was not possible to evaluate the potential synergistic effects of Treg cell ablation in this experimental setup, although checkpoint blockade alone seemed to have a potential effect based on the single checkpoint blockade result (FIG. 7C). These observations suggest that efficient targeting of Treg cells is sufficient and necessary to achieve an effective immunotherapeutic response to the growing tumor in this model of oncogene-dependent cancer.

### Example 8

### Transient Regulatory T-Cell Ablation Significantly Improves the Outcome of Ionizing Radiation Therapy

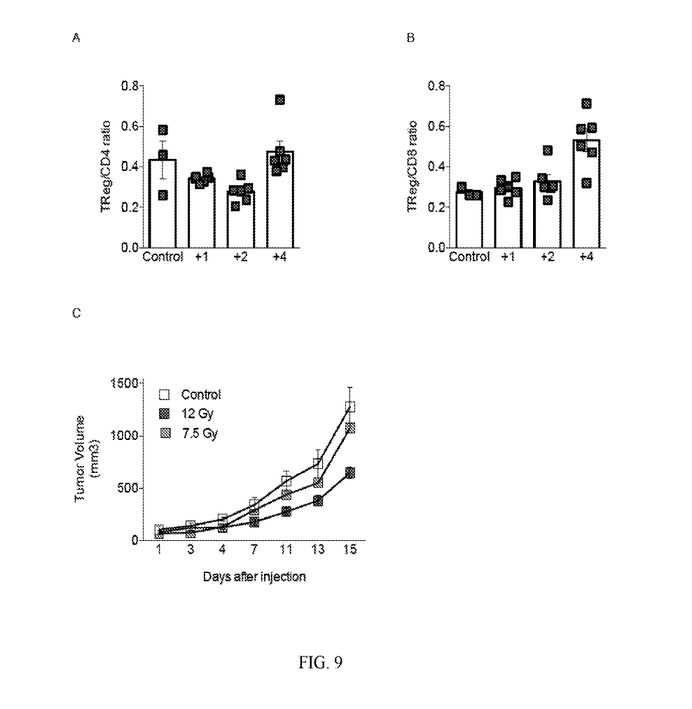

Given that no advantage was derived from combination with checkpoint blockade (the leading immune-based strategy in the treatment of primary tumors), the ability of Treg cell ablation to increase efficacy of ionizing radiation (IR) was explored next. Ionizing radiation is a classic therapeutic strategy aimed at inhibiting proliferation and inducing cell death in tumors. Local radiotherapy, widely used in the management of breast cancer, has the potential to synergize with the observed effects of Treg cell ablation in several ways. First and foremost, Treg cells are markedly more resistant to radiation than conventional T cells, resulting in increasing Treg/T effector cell ratios upon radiotherapy that may reduce its efficacy (see FIG. 9A-B). Secondly, radiation can modulate immune response through the release of tissue damage factors that attract immune cells, stimulate antigen presentation, increase tumor antigen pool and sensitize cancer cells to immune-mediated killing. Lastly, the high rate of cancer cell death resulting in tumor debulking contributes to a decrease in persistent antigens that can induce tolerance.

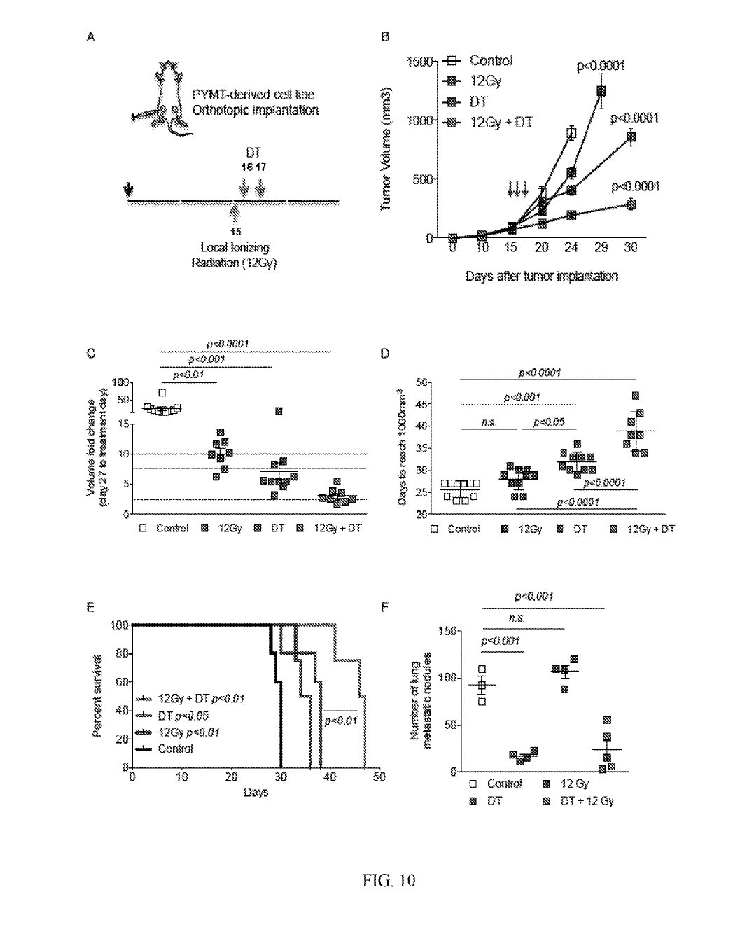

In this study, the effects of both 7.5 Gy and 12 Gy, the two most commonly used doses for local tumor irradiation, was assessed and 12 Gy was used for the remainder of this Example since that regimen reduced the size of the tumors in a 50% by 2 weeks after treatment (FIG. 9C). Next, stereotactic radiation was administered to mice bearing ˜100 mm³ (and 250 mm³, data not shown) bilateral tumors, and depleted Treg cells by administering DT on day 1 and 2 after radiation, prior to rise in Treg cell/Teffector cell ratios (see FIG. 10A; FIG. 9A-B).

As shown in FIG. 10B, the combination of radiation with transient Treg ablation affected the tumor growth much more significantly that either treatment alone, with the most pronounced cooperative effects observed by the end of the experiment. By that time, control and single therapy groups of mice were euthanized due to heavy tumor burden before tumors in the combination treatment group reached exponential growth phase (see FIG. 10B).

As shown in FIG. 10C, during the first two weeks of the experiment, volumes of control tumors had increased 50-fold, irradiated tumors 10-fold, Treg-depleted tumors 7.5-fold, and tumors treated with the combination had only increased ˜2.5-fold. When the average time tumors needed to reach ˜1000 mm³ was measured, FIG. 10D shows that control tumors reached that size in about 25 days, irradiated tumors in 28 days, Treg-depleted tumors in 32 days, and tumor treated with the combination needed an average of 39 days.

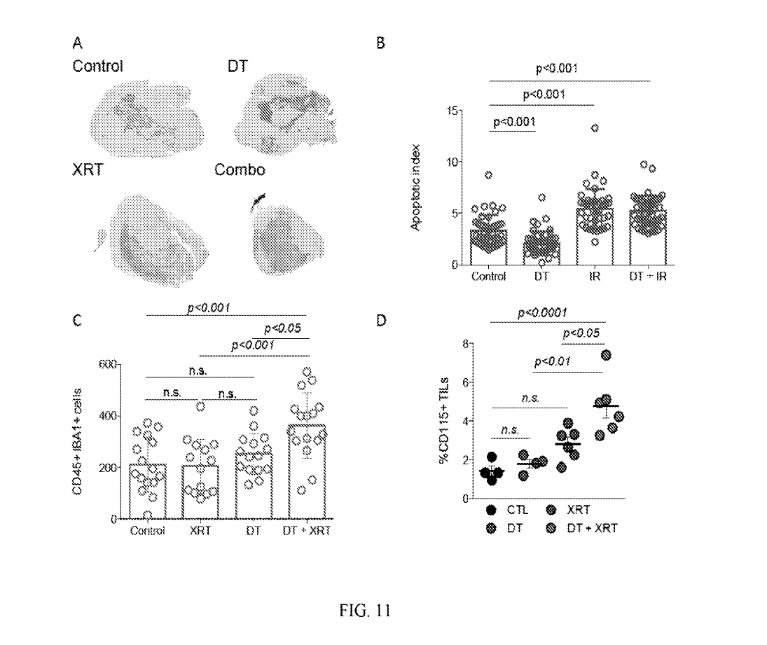

Histological examination of tumors collected from the various groups 2 weeks after treatment showed a significantly bigger area of necrosis in tumors subjected to the a combination treatment than either of single treatments and increased cleaved caspase 3 staining in healthy areas of the tumor (FIG. 11A-B). In addition, tumors in the combination treatment group presented a significant increase in the number of macrophages by double immunohistochemical staining with CD45 and Iba-1 markers (FIG. 11C), as well as increase in CSF-R1⁺ tumor infiltrating leukocytes, as determined by flow cytometry (FIG. 11D). The differences observed in tumor growth translated into a significant increase in mouse survival, with mice treated with the combination therapy living almost twice as long as the control, untreated mice (FIG. 10E). Interestingly, lung metastatic burden analyzed in a time-matched manner was not affected by local radiation treatment, and it was not significantly improved by the combination therapy over the Treg ablation treatment alone, at least at the time of analysis (FIG. 10F), suggesting that transient Treg ablation alone may be effective at limiting distant metastasis.

### Example 9

### Regulatory T-Cell Ablation Reduces Growth of Established Primary Melanoma Tumors

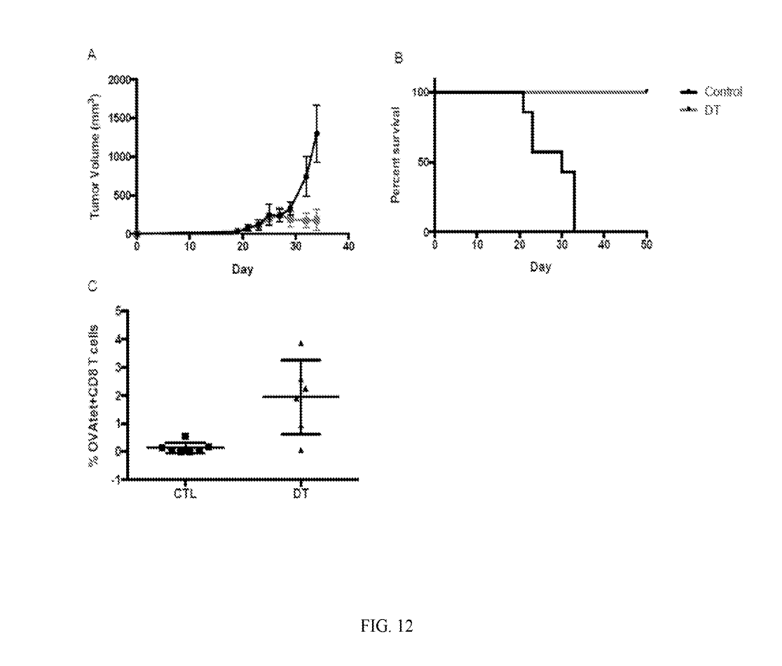

Foxp3-DTR mice with orthotopic implantation of B16-ova melanoma tumors (see Curran et al. PNAS 2010) were evaluated for tumor volume and survival rate in animals with and without Treg ablation. Treg ablation led to reduced growth of tumors (FIG. 12A) and increased mouse survival (FIG. 12B). Flow cytometric analysis showed increased quantities of OVAtet⁺ specific CD8+ in those mice with Treg ablation. The present Example confirms, as demonstrated herein, that Treg cell ablation is therapeutic for melanoma tumors by reducing tumor growth grate and increasing animal survival.

### Example 10

### Regulatory T-Cell Ablation Reduces Growth of Lewis Lung Carcinoma Tumors

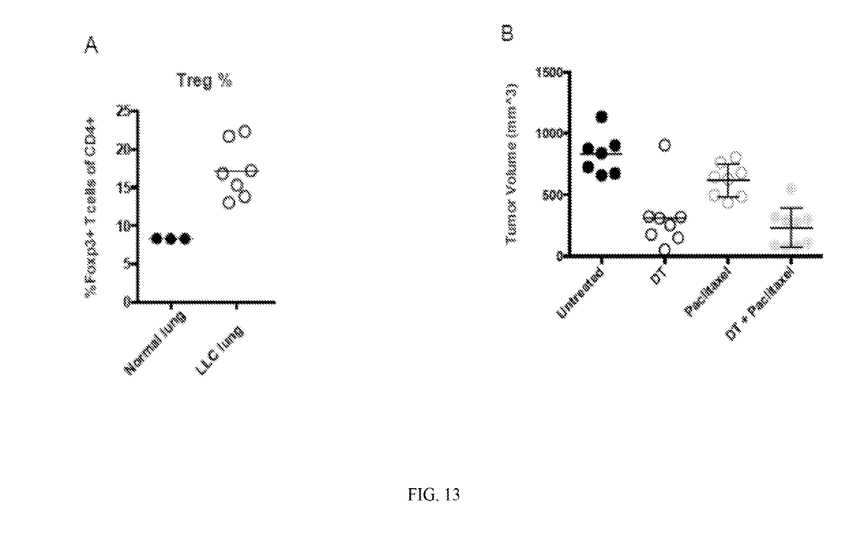

Lewis Lung Carcinoma (LLC) cells were injected intravenously into wild type mice. Tumors that formed in the lung were analyzed 24 days post injection. Flow cytometric analysis demonstrated an influx of Treg cells into lungs of animals with LLC tumors (FIG. 13A). The tumor burden of Foxp3-DTR mice injected with LLC tumor cells was evaluated after Treg depletion alone or in combination with the anti-cancer agent paclitaxel. A reduction in tumor burden was seen both with Treg depletion alone and in combination with paclitaxel (FIG. 13B). The present example further confirms, as demonstrated herein, the role of Foxp3+ T-cells in tumors as well as the ability to reduce tumor burden by Treg depletion alone or in combination with anti-cancer agents.

Observations

The above Examples show that, as a single therapy, checkpoint blockade does not hinder primary tumor progression in a murine orthotopic model of oncogene-driven mammary carcinogenesis. In contrast, efficient ablation of Treg cells alone achieved a significant reduction in tumor burden without the need for additional manipulation, revealing a very significant role of Treg cells in oncogene-driven tumor growth. Treg ablation resulted in a sharply augmented expression of IFN-γ by tumor infiltrating T cells which was necessary for the observed determent of tumor progression. Cytotoxic CD8₊ T cells as well as NK cells were not necessary, whereas CD4⁺ T cells were required for anti-tumor effect of Treg cell mediated ablation (data not shown).

Without wishing to be held to a particular theory, these results indicate that IFN-γ production by NK and CD8⁺ T cells may be dispensable in mediating the anti-tumoral effect of Treg cell ablation, and point to a potential role for CD4⁺ T cells as a non-redundant source of protective IFN-γ in PyMT breast carcinomas. CD4⁺ T cells can exert IFN-γ-dependent as well as direct cytotoxic effects on tumors cells (see Quezada et al., 2010; Shankaran et al., 2001). IFN-γ is known to have pleiotropic activity, and another non-mutually exclusive means by which it may be contributing to the reduction of tumor growth with ablation of Treg cells is by regulating the pro-tumor properties of tumor-infiltrating macrophages. In this regard, the observed sharp increase in expression of iNOS and pro-inflammatory chemokine expression by tumor infiltrating myeloid cells upon Treg cell ablation raises the possibility that the therapeutic effect is secondary to modulation of the accessory functions of tumor-infiltrating macrophages. The latter has been found to be essential for lung metastases in the MMTV-PyMT model (see DeNardo et al., 2009).

In contrast to Treg cell ablation, the anti-tumor effect of both systemic and local administration of anti CTLA-4 antibody is CD8⁺ T cell dependent, but CD4⁺ T cell independent (see Fransen et al., 2013; van Elsas et al., 2001). Likewise, the therapeutic effects of PD-1/PD-L1 blockade in chronic viral infection, and possibly in cancer, are dependent upon restoration of cytolytic responses and IFN-γ production by CD8⁺ T cells (see Barber et al., 2006; Topalian et al., 2012a). The latter in combination with a dispensable role of CD8⁺ T cells for the therapeutic benefit of targeting Treg, may potentially account for the failure of PD-1/PD-L1 blockade to mount independent or additive biological response in the above Examples.

CTLA-4 is expressed by Treg cells and is thought to be required for their function. Genetic studies demonstrated that targeting CTLA-4 in both effector and Treg cell subsets affords the maximal inhibition of tumor growth in a transplantable B16 melanoma model (see Peggs et al., 2009). Considering these findings, the above Example demonstrates the possibility that the success of the PD-1 and CTLA-4 checkpoint blockade may be primarily due to selective (or relative) depletion or functional impairment of Treg cells. This is consistent with recent evidence suggesting that anti-CTLA-4 therapy works primarily through macrophage-mediated Treg ablation (see Selby et al., 2013; J. P. Allison, personal communication). Additionally, PD-1/PD-L1 pathway blockade has also been shown to diminish Treg cell suppressor function (see Wang et al., 2009).

Although CTLA-4 blockade did not affect lung metastatic burden, PD-1/PD-L1 blockage significantly diminished the number of metastatic foci in the lungs. This reduction, albeit markedly less pronounced than the one achieved through Treg cell ablation, is suggestive of a specific role for PD-1/PD-L1 inhibitory pathway in the colonization of lungs by disseminated single cancer cells. The observed selective role for PD-1 in lung metastasis was consistent with its prominent role in blocking lung inflammation, i.e. pneumonitis resulting from PD-1/PD-L1 deficiency, and clinical responses of PD-1 blockade in non-small cell lung cancer patients.

Immune therapeutic approaches such as checkpoint blockade and Treg depletion can lead to the breaking of immune self-tolerance, inducing a variety of side effects that include rash, colitis, hepatitis, and endocrinopathies (see Postow et al., 2012). Moreover, complete and sustained ablation of Treg cells may lead to fatal immune-proliferative syndrome (see Kim et al., 2009; Kim et al., 2007). In these Examples, it is shown that reducing the DT treatment to accomplish efficient, but transient Treg ablation does not have a significant effect on overall mouse morbidity, as evidenced by monitoring mouse activity and weight, and minimizes the immune pathology to very low levels.

Without wishing to be held to a particular theory, the above Examples suggest that targeting Treg cells is likely to result in pronounced clinical responses in breast cancer patients. Modulating this central mechanism of immune tolerance may expand the use of immunotherapy for tumor types that are not inherently immunogenic such as breast cancer. Furthermore, current clinical outcomes might be significantly improved by combination of Treg depletion strategies with radiation, and possibly chemotherapy or targeted therapies against molecular drivers of oncogenesis.

1. A method of treating cancer comprising ablating regulatory t-cells (T_{reg}) in a subject who is suffering from or susceptible to cancer.

2. The method of claim 1, wherein the step of ablating comprises administering a T_{reg} ablating agent.

3. The method of claim 2, wherein the Treg ablating agent is a CCR4 antibody.

4. The method of claim 1, wherein greater than 90% of the T_{reg} in the subject are ablated.

5. The method of claim 1, wherein greater than 95% of the T_{reg} in the subject are ablated.

6. The method of claim 1, wherein greater than 99% of the T_{reg} in the subject are ablated.

7. The method of any one of claims 1 to 6, wherein the step of ablating comprises administering at least two doses of a T_{reg} ablating agent.

8. The method of claim 7, wherein a period of time separates administration of each of the at least two doses of a T_{reg} ablating agent.

9. The method of claim 8, wherein the step of ablating comprises administering at least three doses of a T_{reg} ablating agent, wherein the period of time between each administration is the same.

10. The method of claim 9, wherein the period of time between each administration is different.

11. The method of claim 7, wherein each dose of a T_{reg} ablating agent is the same.

12. The method of any one of claims 1 to 10, wherein the T_{reg} in the subject are ablated for at least 1 day.

13. The method of any one of claims 1 to 12, wherein the Treg are Foxp3+ t-cells.

14. The method of any one of claims 1 to 13, wherein ablation results in a reduction of tumor burden of at least 30%.

15. The method of any one of claims 1 to 13, wherein ablation results in a reduction of tumor burden of at least 50%.

16. The method of any one of claims 1 to 13, wherein ablation results in a reduction of tumor burden of at least 70%.

17. The method of any one of claims 1 to 16, wherein the cancer is metastatic.

18. The method of any one of claims 1 to 17, wherein the cancer is a solid tumor.

19. The method of any one of claims 1 to 17, wherein the cancer comprises a primary tumor.

20. The method of any one of claims 1 to 17, wherein the cancer comprises a secondary tumor.

21. The method of any one of claims 1 to 17, wherein the cancer is selected from the group consisting of: breast cancer, prostate cancer, melanoma, renal cell carcinoma, non-small cell lung cancer, and ovarian cancer.

22. The method of any one of claims 1 to 21, further comprising administering to the subject one or more of: an anticancer agent and ionizing radiation.

23. The method of claim 22, wherein the anti-cancer agent is an anti-CTLA4 agent, an anti-PD-1 agent, and/or an anti-PD-L1 agent.

24. The method of claim 22, wherein the anti-cancer agent is selected from the group consisting of: tamoxifen, anastrozole, letrozole, paclitaxel and trastuzumab.

25. The method of claim 22, wherein between about 5 Gy and about 15 Gy of ionizing radiation is administered.

26. The method of claim 22, wherein between about 7.5 Gy and about 12 Gy of ionizing radiation is administered.

27. The method of any one of claims 1 to 26, wherein the ablation of T_{reg} results in at least one symptom or feature of cancer being reduced in intensity, severity, duration, or frequency, and/or has delayed in onset.
